(19) United States
(12) Patent Application Publication
(10) Pub. No.: US 2010/0152208 A1
(43) Pub. Date: Jun. 17, 2010

(54) BICYCLIC HETEROAROMATIC COMPOUNDS AS INHIBITORS OF STEAROYL-COENZYME A DELTA-9 DESATURASE

(75) Inventors: Serge Leger, Notre-Dame-de-l'ile-Perrot (CA); Denis Deschenes, Dorval (CA); Rejean Fortin, Montreal (CA); Elise Isabel, Pointe-Claire (CA); David Powell, Verdun (CA)

Correspondence Address: MERCK, P O BOX 2000, RAHWAY, NJ, 07065-0907, US

(73) Assignee: MERCK FROSST CANADA LTD.

(21) Appl. No.: 12/600,484

(22) PCT Filed: May 22, 2008
(86) PCT No.: PCT/CA08/00981

§ 371 Date: Nov. 17, 2009

Related U.S. Application Data
(60) Provisional application No. 60/931,460, filed on May 23, 2007.

(51) Int. Cl. A61K 31/519; C07D 513/04; A61P 3/10; A61P 3/06; A61P 9/10

(52) U.S. Cl. 514/260.1; 544/255

(57) ABSTRACT

Bicyclic heteroaromatic compounds of structural formula I are inhibitors of stearoyl-coenzyme A delta-9 desaturase (SCD). The compounds of the present invention are useful for the prevention and treatment of conditions related to abnormal lipid synthesis and metabolism, including cardiovascular disease, such as atherosclerosis; obesity; Type 2 diabetes; insulin resistance; hyperglycemia; Metabolic Syndrome; neurological disease; cancer; and liver steatosis. Formula (I).

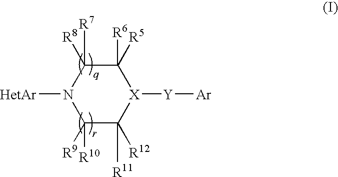

### FIELD OF THE INVENTION

The present invention relates to bicyclic heteroaromatic compounds which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease; atherosclerosis; obesity; diabetes; neurological disease; metabolic syndrome; insulin resistance; cancer; liver steatosis; and non-alcoholic steatohepatitis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCD's) catalyze the rate-limiting formation of the cis-double bond at the C₉-C₁₀ position in monounsaturated fatty acyl-CoAs. The preferred substrates are stearoyl-CoA and palmitoyl-CoA, with the resulting oleoyl and palmitoleoyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS, 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2 or SCD5)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCD's in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD gene (Zheng et al., Nature Genetics, 23: 268-270 (1999)), b) SCD-null mice from targeted gene deletion (Ntambi, et al., PNAS, 99: 11482-11486 (2002), and c) the suppression of SCD expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. SCD knock-out mice (−/−) are characterized by reduced adiposity and increased energy expenditure. Thus, SCD inhibition represents a novel therapeutic strategy in the treatment of Type 2 diabetes, obesity, and related metabolic disorders, such as the Metabolic Syndrome.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). The postprandial de novo lipogenesis is significantly elevated in obese subjects (Marques-Lopes, et al., American Journal of Clinical Nutrition, 73: 252-261 (2001)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim Biophys. Acta, 1486: 285-292 (2000)), and a series of heterocyclic derivatives disclosed in published international patent application publications: WO 2005/011653; WO 2005/011654; WO 2005/011656; WO 2005/011657; WO 2006/014168; WO 2006/034279; WO 2006/034312; WO 2006/034315; WO 2006/034338; WO 2006/034341; WO 2006/034440; WO 2006/034441; WO 2006/034446; WO 2006/086445; WO 2006/086447; WO 2006/101521; WO 2006/125178; WO 2006/125179; WO 2006/125180; WO 2006/125181; WO 2006/125194; WO 2007/044085; WO 2007/046867; WO 2007/046868; WO 2007/050124; WO 2007/130075; and WO 2007/136746, all assigned to Xenon Pharmaceuticals, Inc. A number of international patent applications assigned to Merck Frosst Canada Ltd. that disclose SCD inhibitors useful for the treatment of obesity and Type 2 diabetes have also published: WO 2006/130986 (14 Dec. 2006); WO 2007/009236 (25 Jan. 2007); WO 2007/038865 (12 Apr. 2007); WO 2007/056846 (24 May 2007); WO 2007/071023 (28 Jun. 2007); WO 2007/134457 (29 Nov. 2007); WO 2007/143823 (21 Dec. 2007); and WO 2007/143824 (21 Dec. 2007). WO 2008/003753 (assigned to Novartis) discloses a series of pyrazolo[1,5-a]pyrimidine analogs as SCD inhibitors, and WO 2007/143597 (assigned to Novartis and Xenon Pharmaceuticals) discloses heterocyclic derivatives as SCD inhibitors. Small molecule SCD inhibitors have also been described by G. Liu, et al., “Discovery of Potent, Selective, Orally Bioavailable SCD1 Inhibitors,” in J. Med. Chem., 50: 3086-3100 (2007) and by H. Zhao, et al., “Discovery of 1-(4-phenoxypiperidin-1-yl)-2-arylaminoethanone SCD 1 inhibitors,” Bioorg. Med. Chem. Lett., 17: 3388-3391 (2007).

The present invention is concerned with novel heteroaromatic compounds as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, hyperglycemia, Type 2 diabetes, Metabolic Syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobryzn and J. M. Ntambi, in “Stearoyl-CoA desaturase as a new drug target for obesity treatment,” Obesity Reviews, 6: 169-174 (2005).

### SUMMARY OF THE INVENTION

The present invention relates to bicyclic heteroaromatic compounds of structural formula I:

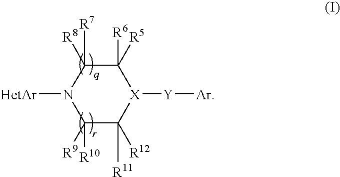

These bicyclic heteroaromatic compounds are effective as inhibitors of SCD. They are therefore useful for the treatment, control or prevention of disorders responsive to the inhibition of SCD, such as diabetes, insulin resistance, lipid disorders, obesity, atherosclerosis, and metabolic syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, insulin resistance, obesity, lipid disorders, atherosclerosis, and metabolic syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating metabolic syndrome by administering the compounds of the present invention in combination with a therapeutically effective amount of another agent known to be useful to treat the condition.

### DETAILED DESCRIPTION OF THE INVENTION

The present invention is concerned with bicyclic heteroaromatic compounds useful as inhibitors of SCD. Compounds of the present invention are described by structural formula I:

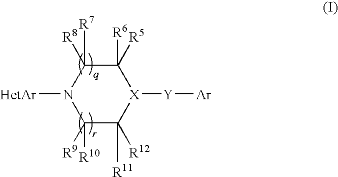

and pharmaceutically acceptable salts thereof; wherein
HetAr is a fused heteroaromatic ring selected from the group consisting of:

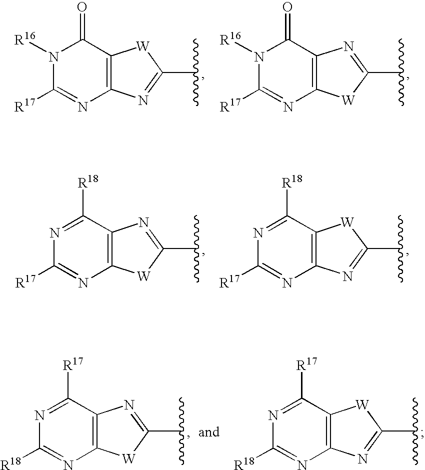

q is 0 or 1;
r is 0 or 1;

### W is O, S, or NR¹⁵;

### X—Y is N—C(O), CR¹⁴—O, CR¹⁴—S(O)₀₋₂, or CR¹³—CR¹R²;

Ar is phenyl, naphthyl, or heteroaryl optionally substituted with one to five R³ substituents;
R¹ and R² are each independently hydrogen or C₁₋₃ alkyl, wherein alkyl is optionally substituted with one to three substituents independently selected from fluorine and hydroxy;
each R³ is independently selected from the group consisting of:

C₁₋₆ alkyl,

C₂₋₆ alkenyl,

(CH₂)ₙ-phenyl,

(CH₂)ₙ-naphthyl,

(CH₂)ₙ-heteroaryl,

(CH₂)ₙ-heterocyclyl,

(CH₂)ₙC₃₋₇ cycloalkyl,

halogen,

nitro,

(CH₂)ₙOR⁴,

(CH₂)ₙN(R⁴)₂,

(CH₂)ₙC≡N,

(CH₂)ₙCO₂R⁴,

(CH₂)ₙNR⁴SO₂R⁴

(CH₂)ₙSO₂N(R⁴)₂,

(CH₂)ₙS(O)₀₋₂R⁴,

(CH₂)ₙNR⁴C(O)N(R⁴)₂,

(CH₂)ₙC(O)N(R⁴)₂,

(CH₂)ₙNR⁴C(O)R⁴,

(CH₂)ₙNR⁴CO₂R⁴,

(CH₂)ₙC(O)R⁴,

O(CH₂)ₙC(O)N(R⁴)₂,

(CH₂)ₛ-Z-(CH₂)ₜ-phenyl,

(CH₂)ₛ-Z-(CH₂)ₜ-naphthyl,

(CH₂)ₛ-Z-(CH₂)ₜ-heteroaryl,

(CH₂)ₛ-Z-(CH₂)ₜ-heterocyclyl,

(CH₂)ₛ-Z-(CH₂)ₜ—C₃₋₇ cycloalkyl,

(CH₂)ₛ-Z-(CH₂)ₜ—OR⁴,

(CH₂)ₛ-Z-(CH₂)ₜ—N(R⁴)₂,

(CH₂)ₛ-Z-(CH₂)ₜ—NR⁴SO₂R⁴,

(CH₂)ₛ-Z-(CH₂)ₜ—C≡N,

(CH₂)ₛ-Z-(CH₂)ₜ—CO₂R⁴,

(CH₂)ₛ-Z-(CH₂)ₜ—SO₂N(R⁴)₂,

(CH₂)ₛ-Z-(CH₂)ₜ—S(O)₀₋₂R⁴,

(CH₂)ₛ-Z-(CH₂)ₜ—NR⁴C(O)N(R⁴)₂,

(CH₂)ₛ-Z-(CH₂)ₜ—C(O)N(R⁴)₂,

(CH₂)ₛ-Z-(CH₂)ₜ—NR⁴C(O)R⁴,

(CH₂)ₛ-Z-(CH₂)ₜ—NR⁴CO₂R⁴,

(CH₂)ₛ-Z-(CH₂)ₜ—C(O)R⁴,

CF₃,

CH₂CF₃,

OCF₃, and

OCH₂CF₃;

in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;

### Z is O, S, or NR⁴;

each R⁴ is independently selected from the group consisting of

hydrogen,

C₁₋₆ alkyl,

(CH₂)ₘ-phenyl,

(CH₂)ₘ-heteroaryl,

(CH₂)ₘ-naphthyl, and

(CH₂)ₘC₃₋₇ cycloalkyl;

wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
R⁵, R⁶, R⁷, R⁸, R⁹, R¹⁰, R¹¹, and R¹² are each independently hydrogen, fluorine, or C₁₋₃ alkyl, wherein alkyl is optionally substituted with one to three substituents independently selected from fluorine and hydroxy;
R¹³ is hydrogen, C₁₋₃ alkyl, fluorine, or hydroxy;
each R¹⁴ is independently hydrogen or C₁₋₃ alkyl;
R¹⁵ is selected from the group consisting of hydrogen, C₁₋₄ alkyl, C₁₋₄ alkylcarbonyl, aryl-C₁₋₂ alkylcarbonyl, arylcarbonyl, C₁₋₄ alkylaminocarbonyl, C₁₋₄ alkylsulfonyl, arylsulfonyl, aryl-C₁₋₂ alkylsulfonyl, C₁₋₄ alkyloxycarbonyl, aryloxycarbonyl, and aryl-C₁₋₂ alkyloxycarbonyl;
R¹⁶ is hydrogen or C₁₋₃ alkyl optionally substituted with one to five fluorines;
R¹⁷ is selected from the group consisting of:

—(CH₂)ᵥC(O)R^{a},

—(CH₂)_{y}-T-(CH₂)_{z}C(O)R^{a},

—(CH₂)_{y}-T-(CH₂)_{z}SO₃H,

—(CH₂)_{y}-T-(CH₂)_{w}-phenyl,

—(CH₂)_{y}-T-(CH₂)_{w}-heteroaryl,

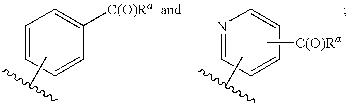

wherein phenyl and heteroaryl are optionally substituted with one to two substituents independently selected from halogen, C₁₋₄ alkyl, —(CH₂)_{X}C(O)R^{a}, and —CH═CHC(O)R^{a}; wherein any methylene (CH₂) carbon atom in R¹⁷ is optionally substituted with one to two groups independently selected from amino, carboxy, fluorine, hydroxy, and C₁₋₄ alkyl; or two substituents when on the same methylene (CH₂) group are taken together with the carbon atom to which they are attached to form a cyclopropyl group;

### T is O, S, or NR¹⁴;

R^{a} is OH, —OC₁₋₄ alkyl, —NH₂, —NHSO₂C₁₋₄ alkyl, —NHSO₂C₃₋₆ cycloalkyl, or —NHSO₂CH₂C₃₋₆ cycloalkyl;
R¹⁸ is selected from the group consisting of:
amino,
halogen,
C₁₋₄ alkoxy, optionally substituted with hydroxy or carboxy,
C₁₋₄ alkylthio, optionally substituted with hydroxy or carboxy,
C₁₋₄ alkylamino,
di-(C₁₋₄ alkyl)amino,
arylamino,
aryl-C₁₋₂ alkylamino,
C₁₋₄ alkylcarbonylamino,
aryl-C₁₋₂ alkylcarbonylamino,
arylcarbonylamino,
C₁₋₄ alkylaminocarbonylamino,
C₁₋₄ alkylsulfonylamino,
arylsulfonylamino,
aryl-C₁₋₂ alkylsulfonylamino,
C₁₋₄ alkyloxycarbonylamino,
aryloxycarbonylamino, and
aryl-C₁₋₂ alkyloxycarbonylamino;
   each m is independently an integer from 0 to 2;
   each n is independently an integer from 0 to 2;
   each s is independently an integer from 1 to 3;
   each t is independently an integer from 1 to 3;
   v is an integer from 0 to 4;
   w is an integer from 0 to 2;
   z is 1 or 2;
   each x is an integer from 0 to 2; and
   each y is 0 or 1.

In one embodiment of the compounds of the present invention, q and r are both 1, affording a 6-membered piperidine ring.

In a second embodiment of the compounds of the present invention, q is 1 and r is 0, affording a 5-membered pyrrolidine ring.

In a third embodiment of the compounds of the present invention, q and r are both 0, affording a 4-membered azetidine ring.

In a fourth embodiment of the compounds of the present invention, X—Y is N—C(O). In a class of this embodiment, Ar is phenyl substituted with one to three R³ substituents as defined above.

In a fifth embodiment of the compounds of the present invention, X—Y is CR¹⁴—O. In a class of this embodiment, R¹⁴ is hydrogen and Ar is phenyl substituted with one to three R³ substituents as defined above.

In a sixth embodiment of the compounds of the present invention, X—Y is CR¹⁴—S. In a class of this embodiment, R¹⁴ is hydrogen and Ar is phenyl substituted with one to three R³ substituents as defined above.

In a seventh embodiment of the compounds of the present invention, X—Y is CR¹³—CR¹R². In a class of this embodiment, R¹, R², and R¹³ are each hydrogen and Ar is phenyl substituted with one to three R³ substituents as defined above.

In an eighth embodiment of the compounds of the present invention, R⁵, R⁶, R⁷, R⁸, R⁹, R¹⁰, R¹¹, and R¹² are each hydrogen.

In a ninth embodiment of the compounds of the present invention, HetAr is

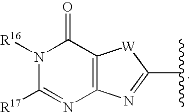

In a class of this embodiment, W is S. In a subclass of this class, R¹⁶ is hydrogen.

In a second class of this ninth embodiment of the compounds of the present invention, R¹⁷ is —(CH₂)ᵥC(O)R^{a} wherein R^{a} is —OH or —OC₁₋₄ alkyl and v is an integer from 1 to 3. In a subclass of this class, v is 2.

In a third class of this embodiment of the compounds of the present invention, R¹⁷ is —(CH₂)_{y}—S—(CH₂)C(O)R^{a} wherein R^{a} is —OH or —OC₁₋₄ alkyl and y is as defined above.

In a fourth class of this ninth embodiment of the compounds of the present invention, R¹⁷ is —(CH₂)_{y}-T-(CH₂)_{w}-pyridyl or —(CH₂)_{y}-T-(CH₂)_{w}-phenyl wherein

y is 0 or 1;

w is 0 or 1;

T is O or S; and

phenyl and pyridyl are substituted with one substituent selected from —(CH₂)_{X}C(O)R^{a} and —CH═CHC(O)R^{a} wherein R^{a} is —OH or —OC₁₋₄ alkyl and x is as defined above.

In a tenth embodiment of the compounds of the present invention, Ar is phenyl substituted with one to two substituents independently selected from the group consisting from C₁₋₄ alkyl, halogen, CF₃, and phenyl optionally substituted with one to two substituents independently selected from the group consisting of halogen, hydroxy, C₁₋₄ alkyl, trifluoromethyl, and C₁₋₄ alkoxy.

A further embodiment of the present invention relates to compounds of structural formula (II):

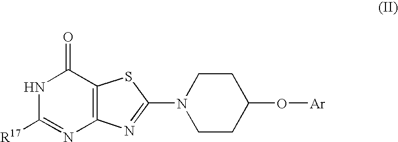

wherein

Ar is phenyl substituted with one to two substituents independently selected from the group consisting from C₁₋₄ alkyl, halogen, CF₃, and phenyl optionally substituted with one to two substituents independently selected from the group consisting of halogen, hydroxy, C₁₋₄ alkyl, trifluoromethyl, and C₁₋₄ alkoxy;

R¹⁷ is selected from the group consisting of

—(CH₂)ᵥC(O)R^{a},

—(CH₂)_{y}—S—CH₂C(O)R^{a},

(CH₂)_{y}-T-(CH₂)_{w}-pyridyl, and

—(CH₂)_{y}-T-(CH₂)_{w}-phenyl;

T is O or S; and

phenyl and pyridyl are substituted with one substituent selected from —(CH₂)_{X}C(O)R^{a} and —CH═CHC(O)R^{a}; and wherein R^{a} is —OH or —OC₁₋₄ alkyl; v is an integer from 1 to 3; y is 0 or 1; w is 0 or 1; and x is an integer from 0 to 2.

Yet a further embodiment of the present invention relates to compounds of structural formula (III):

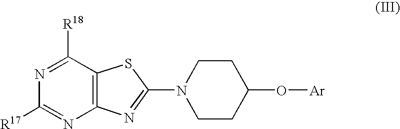

wherein

Ar is phenyl substituted with one to two substituents independently selected from the group consisting from C₁₋₄ alkyl, halogen, CF₃, and phenyl optionally substituted with one to two substituents independently selected from the group consisting of halogen, hydroxy, C₁₋₄ alkyl, trifluoromethyl, and C₁₋₄ alkoxy;

R¹⁸ is selected from the group consisting of

amino,

halogen,

C₁₋₄ alkoxy, optionally substituted with hydroxy or carboxy,

C₁₋₄ alkylthio, optionally substituted with hydroxy or carboxy,

C₁₋₄ alkylamino, and

di-(C₁₋₄ alkyl)amino;

R¹⁷ is selected from the group consisting of

—(CH₂)ᵥC(O)R^{a},

—(CH₂)_{y}—S—CH₂C(O)R^{a},

—(CH₂)_{y}-T-(CH₂)_{w}-pyridyl, and

—(CH₂)_{y}-T-(CH₂)_{w}-phenyl;

T is O or S; and

phenyl and pyridyl are substituted with one substituent selected from —(CH₂)_{X}C(O)R^{a} and —CH═CHC(O)R^{a}; and wherein R^{a} is —OH or —OC₁₋₄ alkyl; v is an integer from 1 to 3; y is 0 or 1; w is 0 or 1; and x is an integer from 0 to 2.

Illustrative, but nonlimiting examples, of compounds of the present invention that are useful as inhibitors of SCD are the following:

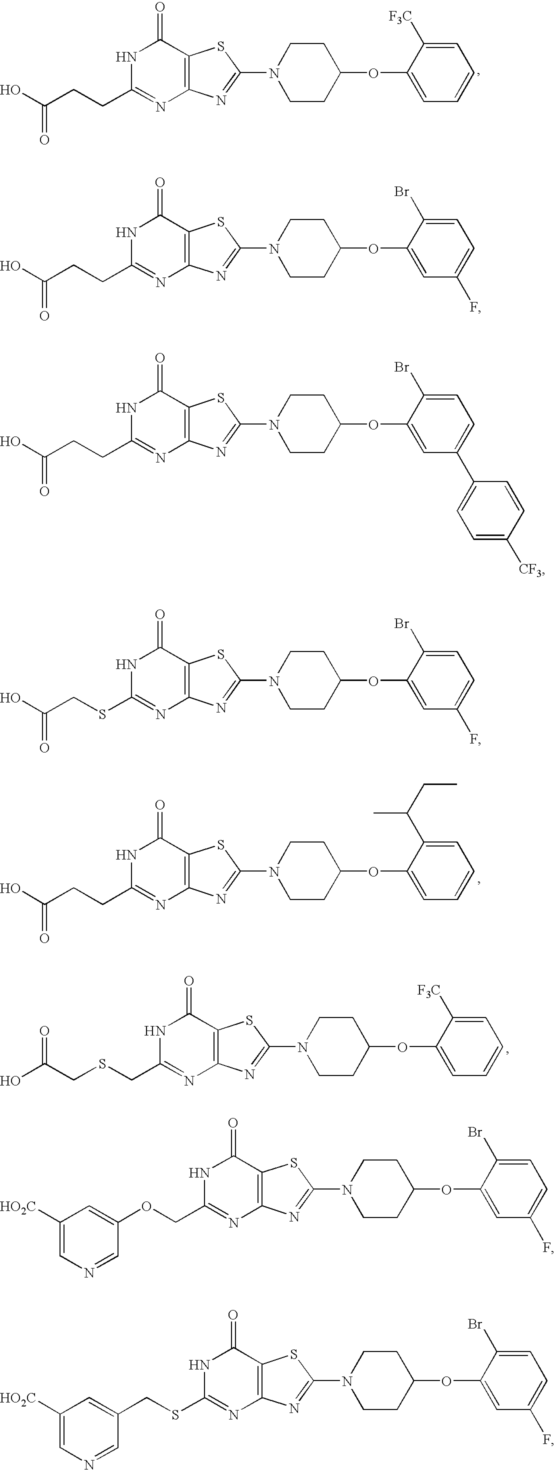

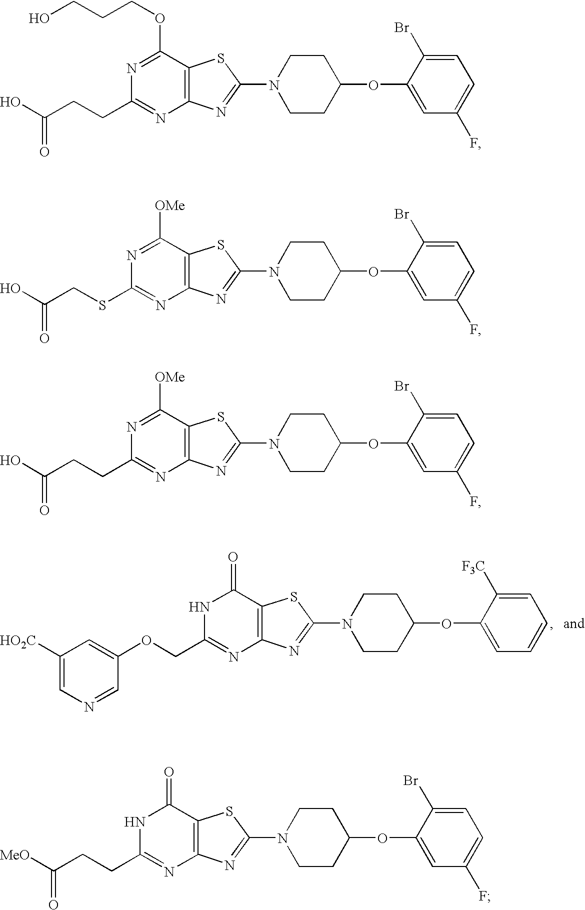

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

By “carboxy” is meant the residue —COOH.

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention. Examples of tautomers which are intended to be encompassed within the compounds of the present invention are illustrated below:

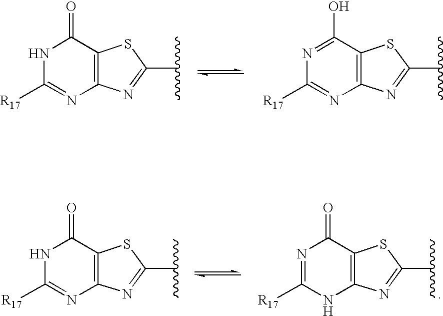

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer. In one embodiment of this aspect of the invention, the cancer is liver cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of and or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD-Induced Rat Liver Microsome Assay:

The activity of compounds of formula I against the SCD enzyme is determined by following the conversion of radiolabeled-stearoyl-CoA to oleoyl-CoA using SCD-induced rat liver microsome and a previously published procedure with some modifications (Joshi, et al., J. Lipid Res., 18: 32-36 (1977)). After feeding wistar rats with a high carbohydrate/fat-free rodent diet (LabDiet # 5803, Purina) for 3 days, the SCD-induced livers were homogenized (1:10 w/v) in 250 mM sucrose, 1 mM EDTA, 5 mM DTT and 50 mM Tris-HCl (pH 7.5). After a 20 min centrifugation (18,000×g/4° C.) to remove tissue and cell debris, the microsome was prepared by a 100,000×g centrifugation (60 min) with the resulting pellet suspended in 100 mM sodium phosphate, 20% glycerol and 2 mM DTT. Test compound in 2 μL DMSO was incubated for 15 min. at room temperature with 180 μL, of the microsome (typically at about 100 μg/mL, in Tris-HCl buffer (100 mM, pH 7.5), ATP (5 mM), Coenzyme A (0.1 mM), Triton X-100 (0.5 mM) and NADH (2 mM)). The reaction was initiated by the addition of 20 μL of [³H]-Stearoyl-CoA (final concentration at 2 μM with the radioactivity concentration at 1 μCi/mL), and terminated by the addition of 150 μL of 1N sodium hydroxide. After 60 min at room temperature to hydrolyze the oleoyl-CoA and stearoyl-CoA, the solution was acidified by the addition of 150 μL of 15% phosphoric acid (v/v) in ethanol supplemented with 0.5 mg/mL stearic acid and 0.5 mg/mL oleic acid. [³H]-oleic acid and [³H]-stearic acid were then quantified on a HPLC that is equipped with a C-18 reverse phase column and a Packard Flow Scintillation Analyzer. Alternatively, the reaction mixture (80 μL) was mixed with a calcium chloride/charcoal aqueous suspension (100 μL of 15% (w/v) charcoal plus 20 μL of 2 N CaCl₂). The resulting mixture was centrifuged to precipitate the radioactive fatty acid species into a stable pellet. Tritiated water from SCD-catalyzed desaturation of 9,10-[³H]-stearoyl-CoA was quantified by counting 50 μL of the supernant on a scintillation counter.

### II. Whole Cell-Based SCD (Delta-9), Delta-5 and Delta-6 Desaturase Assays:

Human HepG2 cells were grown on 24-well plates in MEM media (Gibco cat# 11095-072) supplemented with 10% heat-inactivated fetal bovine serum at 37° C. under 5% CO₂ in a humidified incubator. Test compound dissolved in the media was incubated with the subconfluent cells for 15 min at 37° C. [1-¹⁴C]-stearic acid was added to each well to a final concentration of 0.05 μCi/mL to detect SCD-catalyzed [¹⁴C]-oleic acid formation. 0.05 μCi/mL of [1-¹⁴C]-eicosatrienoic acid or [1-¹⁴C]-linolenic acid plus 10 μM of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 μL of 2N sodium hydroxide plus 50 μl, of L-α-phosphatidylcholine (2 mg/mL in isopropanol, Sigma #P-3556). After acidification with phosphoric acid (60 μL), the radioactive species were extracted with 300 μL of acetonitrile and quantified on a HPLC that was equipped with a C-18 reverse phase column and a Packard Flow Scintillation Analyzer. The levels of [¹⁴ C]-oleic acid over [¹⁴C]-stearic acid, [¹⁴C]-arachidonic acid over [¹⁴C]-eicosatrienoic acid, and [¹⁴C]-eicosatetraenoic acid (8, 11, 14, 17) over [¹⁴C]-linolenic acid were used as the corresponding activity indices of SCD, delta-5 and delta-6 desaturase, respectively.

The SCD inhibitors of formula I, particularly the inhibitors of Examples 1 through 43, exhibit an inhibition constant IC₅₀ of less than 1 μM and more typically less than 0.1 μM. Generally, the IC₅₀ ratio for delta-5 or delta-6 desaturases to SCD for a compound of formula I, particularly for Examples 1 through 43, is at least about ten or more, and preferably about hundred or more.

### In Vivo Efficacy of Compounds of the Present Invention:

The in vivo efficacy of compounds of formula I was determined by following the conversion of [1-¹⁴C]-stearic acid to [1-¹⁴C]oleic acid in animals as exemplified below. Mice were dosed with a compound of formula I and one hour later the radioactive tracer, [1-¹⁴C]-stearic acid, was dosed at 20 μCi/kg IV. At 3 h post dosing of the compound, the liver was harvested and then hydrolyzed in 10 N sodium hydroxide for 24 h at 80° C., to obtain the total liver fatty acid pool. After phosphoric acid acidification of the extract, the amount of [1-¹⁴C]-stearic acid and [1-¹⁴C]-oleic acid was quantified on a HPLC that was equipped with a C-18 reverse phase column and a Packard Flow Scintillation Analyzer.

The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of formula I, and either administered separately or in the same pharmaceutical composition, include, but are not limited to:

(a) dipeptidyl peptidase IV (DPP-IV) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), melanin-concentrating hormone (MCH) receptor antagonists, and microsomal triglyceride transfer protein (MTP) inhibitors;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators; and

(v) oxyntomodulin and derivatives and analogs thereof.

Dipeptidyl peptidase-IV inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-IV inhibitor compounds include sitagliptin (MK-0431); NVP-DPP-728; vildagliptin (LAF 237); P93/01; alogliptin (SYR-322); denagliptin; and saxagliptin (BMS 477118).

Antiobesity compounds that can be combined with compounds of structural formula I include fenfluramine, dexfenfluramine, phentermine, sibutramine, orlistat, neuropeptide Y₁ or Y₅ antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds of formula I include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634. Specific cannabinoid CB! receptor antagonists include rimonabant and taranabant.

Melanocortin-4 receptor (MC4R) agonists useful in the present invention include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02/092566, WO 03/-093234, WO 03/095474, and WO 03/104761.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

In another aspect of the invention, a pharmaceutical composition is disclosed which comprises:

(1) a compound of structural formula I;
(2) one or more compounds selected from the group consisting of:

(a) dipeptidyl peptidase IV (DPP-IV) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), melanin-concentrating hormone (MCH) receptor antagonists, and microsomal triglyceride transfer protein (MTP) inhibitors;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib; and

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors; and

(u) AMPK activators; and

(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Preparation of Compounds of the Invention:

The compounds of structural formula (I) can be prepared according to the procedures of the following Schemes and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESMS).

### List of Abbreviations:

Alk=alkyl
APCI=atmospheric pressure chemical ionization
Ar=aryl
Boc=tert-butoxycarbonyl
br=broad
Cbz=benzyloxycarbonyl
CH₂Cl₂=dichloromethane
CH₂N₂=diazomethane
d=doublet
DBU=1,8-diazabicyclo[5.4.0]undec-7-ene
DCC=N,N′-dicyclohexylcarbodiimide
DEAD=diethyl azodicarboxylate
Deoxofluor®=bis(2-methoxyethyl)aminosulfur trifluoride

### DIPEA=N,N-diisopropylethylamine

### DMF=N,N-dimethylformamide

DMSO=dimethyl sulfoxide
ESI=electrospray ionization
EtOAc=ethyl acetate
HATU=O-(7-azabenzotriazol-1-yl)-N,N,N,N′-tetramethyluronium hexafluorophosphate
HOAc=acetic acid
HOBt=1-hydroxybenzotriazole hydrate
KOH=potassium hydroxide
LC-MS=liquid chromatography-mass spectroscopy
LiOH=lithium hydroxide
m=multiplet
m-CPBA=3-chloroperoxybenzoic acid
MeOH=methyl alcohol
MgSO₄=magnesium sulfate
MMPP=magnesium monoperoxyphthalate
MS=mass spectroscopy
NaHMDS=sodium bis(trimethylsilyl)amide
NaOH=sodium hydroxide
Na₂SO₄=sodium sulfate
NH₄OAc=ammonium acetate

### NMP=N-methylpyrrolidinone

NMR=nuclear magnetic resonance spectroscopy
PG=protecting group
rt=room temperature
s=singlet
t=triplet
THF=tetrahydrofuran
TFA=trifluoroacetic acid
TFAA=trifluoroacetic anhydride
TLC=thin-layer chromatography
TsCl=p-toluenesulfonyl chloride
p-TsOH=p-toluenesulfonic acid

### Method A:

Compounds of structural formula (I) wherein X is C can be prepared by Method A. An appropriately substituted and N-protected-4-hydroxypiperidine (1) is first coupled to an ArOH or ArSH unit by a Mitsunobu reaction (see Tanaka, N.; Goto, R.; Ito, R.; Hayakawa, M.; Ogawa, T.; Fujimoto, K. Chem. Pharm. Bull. 1998, 46, 639-646; Fletcher, S. R.; Burkamp, F.; Blurton, P.; Cheng, S. K. F.; Clarkson, R.; O′Connor, D.; Spinks, D.; Tudge, M.; Niel, M. B.; Patel, S.; Chapman, K.; J. Med. Chem. 2002, 45, 492-503; Ohno, K. I.; Fukushima, T.; Santa, T.; Waizumi, N.; Tokuyama, H.; Masako, M.; Imai, K.; Anal. Chem. 2002, 74, 4391-4396). The piperidine nitrogen protecting group (PG) is then cleaved to give 3 which can be elaborated to 4 and then to 5 according to published literature procedures (W=S, O, N; see Ried W.; Kuhnt D. Liebigs. Ann. Chem. 1986, 780-784; McCarty, C. G. et al., J. Org. Chem. 1970, 35, 2067-2069; Gante J.; Mohr G. Chem. Ber. 1975, 108, 174-180, respectively). Treatment of 5 with a suitable base and solvent combination such as triethylamine in methanol affords the 5-membered heteroaromatic ring in 6 via an intramolecular attack of a resonance-stabilized carbanion (G=nitrile, ester or amide when W=S; nitrile when W=O or NR¹⁵) onto the carbon of the cyanamide (see Ried W.; Kuhnt D. Liebigs Ann. Chem. 1986, 780-784). When G=CONH₂, subsequent reaction with an acid chloride in the corresponding carboxylic acid or ester as solvent, affords compounds of the present invention denoted by 7 (Dotsenko, V. V.; Krivokolysko, S. G.; Litvinov, V. P., Chem. Heterocycl. Compd. 2003, 39, 110-112). Alternatively, compounds 7 can be prepared by condensation of 6 with an orthoester in the presence of an acid catalyst such as TsOH. When R¹⁷ of compounds 7 contains an ester functionality, saponification using lithium or sodium hydroxide in a suitable solvent such as aqueous methanol gives the desired free carboxylic acid derivative.

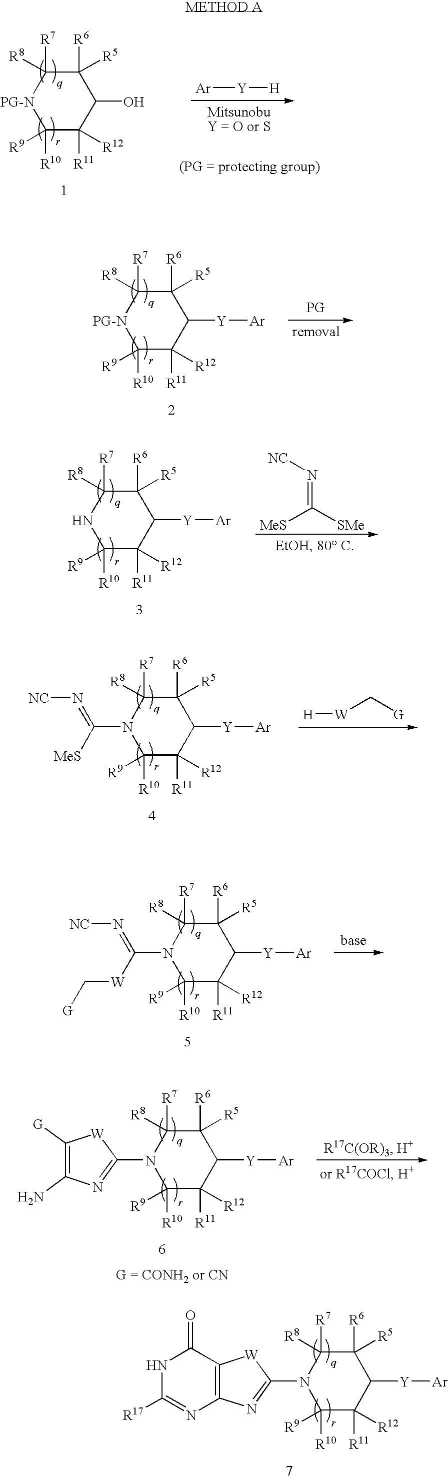

### Method B:

When L is a leaving group, such as halogen or a sulfonate, intermediates 8 can be prepared using procedures described to prepare compounds 7 using the appropriately substituted acetyl chloride in the substituted acetic acid as the solvent. Compounds 9 can be prepared from intermediates 8 by displacement of L with a nucleophile R^{17′}-TH (T=O, S, or N) in the presence of a suitable base. Alternatively, L can be displaced with PG-TH to afford intermediates 10 which upon deprotection can be alkylated with R^{17′}-LG, wherein LG is a leaving group, to give compounds 9. When R^{17′} in compounds 9 contains a carboxylic acid ester functionality, saponification using aqueous lithium or sodium hydroxide in a suitable solvent such as aqueous methanol affords the free carboxylic acid derivative.

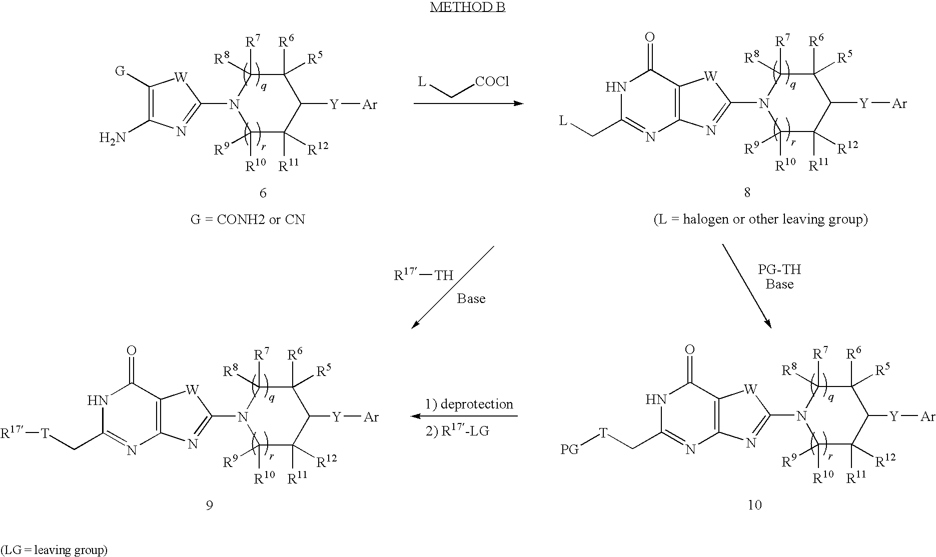

### Method C:

Piperidinol 11 can be elaborated into 12 according to published literature procedures discussed in Method A (W=S, O, N; see Ried W.; Kuhnt D. Liebigs. Ann. Chem. 1986, 780-784; McCarty, C. G. et al., J. Org. Chem. 1970, 35, 2067-2069; Gante J.; Mohr G. Chem. Ber. 1975, 108, 174-180, respectively). For the conversion of 12 into the 5-membered heteroaromatic ring 14, procedures described in Ried W.; Kuhnt D. Liebigs Ann. Chem. 1986, 780-784, can be used. When G=CONH₂, subsequent reaction with an acid chloride in the corresponding carboxylic acid or ester as solvent, affords intermediate 15 wherein the secondary hydroxyl group is acylated. (Dotsenko, V. V.; Krivokolysko, S. G.; Litvinov, V. P., Chem. Heterocycl. Compd. 2003, 39, 110-112). Cleavage of the acyl group is achieved by treatment with sodium methoxide in methanol. Finally, intermediates 16 can be coupled to an ArOH or ArSH unit by a Mitsunobu reaction as described in the first step of Method A to afford compounds 7. When R¹⁷ in compounds 7 contains an ester functionality, saponification using aqueous lithium or sodium hydroxide in a suitable solvent such as aqueous methanol affords the free carboxylic acid derivative.

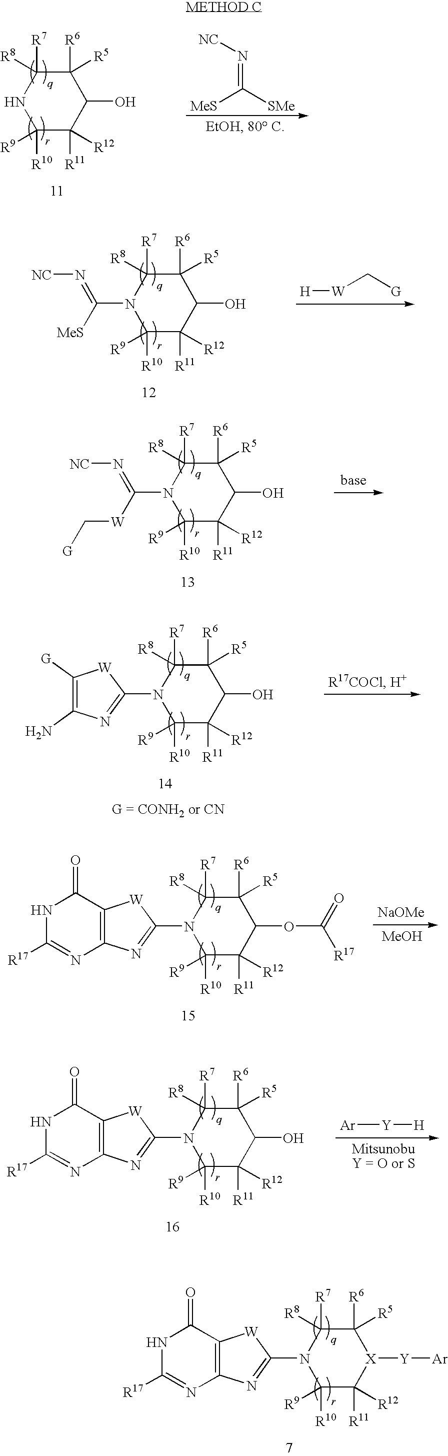

### Method D:

The corresponding pyrimidinones 7 can be converted to the chloropyrimidine 17 with the use of a chlorinating reagent such as thionyl chloride, oxalyl chloride, and phosphorous oxychloride. Final compounds 18 can be prepared from displacement of the chloropyrimidine 17 with an appropriate nucleophile, such as an alcohol, amine, and thiol.

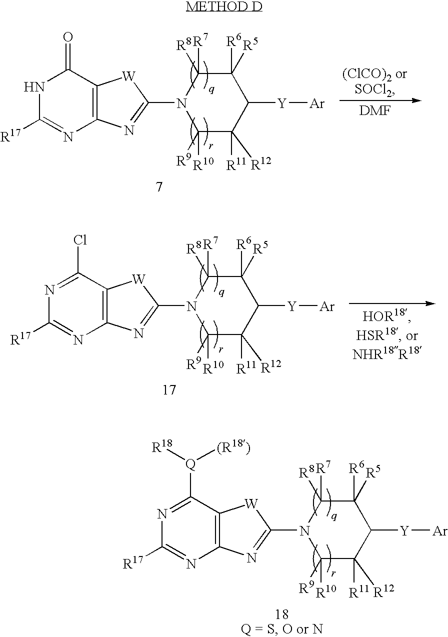

### Example 1

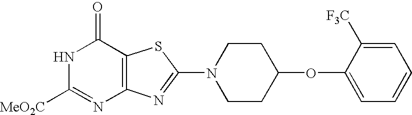

### Methyl 7-oxo-2-{4-[2-(trifluoromethyl)phenoxy]piperidin-1-yl}-6,7-dihydro[1,3]-thiazolo[4,5-d]pyrimidine-5-carboxylate

### Step 1: tert-Butyl 4-[2-(trifluoromethyl)phenoxy]piperidine-1-carboxylate

Diethyl azodicarboxylate (18.9 mL, 120 mmol) was added dropwise to a 0° C. solution of tent-butyl-4-hydroxypiperidine-1-carboxylate (20.13 g, 100 mmol), 2-trifluoromethylphenol (17.83 g, 110 mmol) and triphenylphosphine (31.44 g. 120 mmol) in THF (300 mL). The mixture was then warmed to rt and stirred for 16 h before being concentrated and partitioned between ether and water. The ether phase was washed with 2 M NaOH and water, dried over Na₂SO₄ and concentrated. The residue was then suspended in a mixture of ether and hexanes (35/65) and filtered to remove most of the triphenylphosphine oxide by-product. The filtrate was concentrated and the residue was subjected to flash chromatography on silica gel eluting with 35/65 ether/hexanes to afford the title compound as a colorless solid.

### Step 2: 4-[2-{Trifluoromethyl}phenoxy]piperidine

A solution of tert-butyl 4-[2-(trifluoromethyl)phenoxy]piperidine-1-carboxylate (28.65 g, 83.0 mmol) in CH₂Cl₂ (200 mL) was cooled to 0° C. and treated with trifluoroacetic acid (25.5 mL, 330 mmol) with stirring at rt for 10 h. The reaction mixture was then concentrated and the residue was taken up in ethyl acetate, washed with 2 M NaOH and brine, and the organic phase was dried over Na₂SO₄. Concentration in vacuo and flash chromatography on silica gel eluting with Jan. 9, 1990 NH₄OH/MeOH/CH₂Cl₂ gave the title compound as a faint-yellow syrup.

### Step 3: Methyl N-cyano-[2-(trifluoromethyl)phenoxy]piperidine-1-carbimidothioate

4-[2-{Trifluoromethyl}phenoxy]piperidine (1.12 g, 4.57 mmol) and dimethyl N-cyanodithioiminocarbonate (670 mg, 4.60 mmol) were heated together at reflux temperature in ethanol (1.5 mL) for 30 min The mixture was then concentrated in vacuo to afford the title compound as a thick yellow syrup.

### Step 4: 4-Amino-2-{4-[2-(trifluoromethyl)phenoxy]piperidin-1-yl}-1,3-thiazole-5-carboxamide

Triethylamine (2.0 mL, 15 mmol) was added to a mixture of methyl N-cyano-[2-(trifluoromethyl)phenoxy]piperidine-1-carbimidothioate (1.56 g, 4.57 mmol), 2-mercaptoacetamide (4.2 mL, 4.6 mmol, 10 wt % in methanolic ammonia) and the solution was left to stand at rt overnight after thorough mixing by swirling. The mixture was then cooled to 0° C. and filtered. The solid that was collected was washed with ice cold methanol and dried under vacuum to afford the title compound as a colorless solid.

### Step 5: Methyl 7-oxo-2-{4-[2-(trifluoromethyl)phenoxy]piperidin-1-yl}-6,7-dihydro[1,3]thiazolo[4,5-d]pyrimidine-5-carboxylate

4-Amino-2-{4-[2-(trifluoromethyl)phenoxy]piperidin-1-yl}-1,3-thiazole-5-carboxamide (245 mg, 0.63 mmol) and dimethyl oxalate (995 mg) were heated at 80° C. to give a homogeneous solution. Methyl oxalyl chloride (0.2 mL, 2.2 mmol) was then added dropwise. The resulting yellow-green solution was stirred at 120° C. for 4.5 h. The reaction mixture was allowed to cool to rt and partitioned between EtOAc and water. The organic layer was washed with half saturated NaHCO₃, dried over Na₂SO₄, and concentrated. The crude product was loaded onto silica gel and eluted with a gradient of ethyl acetate in hexanes going from 0% to 100% to afford the title compound as a white solid. ¹H NMR (400 MHz, d₆-acetone): δ 11.3 (bs, 1H), 7.68-7.61 (m, 2H), 7.40 (d, 1H), 7.13 (t, 1H), 5.10-5.05 (m, 1H), 3.99 (s, 3H), 3.88 (m, 4H) 2.26-2.17 (m, 2H), 1.98 (m, 2H) ppm. MS (APCI, m/z 454.9 [M+11]⁺.

### Example 2

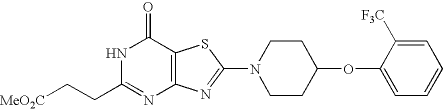

### Methyl 3-(7-oxo-2-{4-[2-(trifluoromethyl)phenoxy]piperidin-1-yl}-6,7-dihydro[1,3]thiazolo[4,5-d]pyrimidin-5-yl)propanoate

To a solution of 4-amino-2-{4-[2-(trifluoromethyl)phenoxy]piperidin-1-yl}-1,3-thiazole-5-carboxamide (245 mg, 0.63 mmol, from Step 4 of Example 1) in dimethyl succinate (1 mL) was added dropwise carbomethoxypropionyl chloride (0.15 mL, 1.2 mmol). The resulting yellow solution was stirred at 125° C. for 4 h. The reaction mixture was allowed to cool to rt and partitioned between EtOAc and aqueous ammonium acetate (25% w/v). The organic layer was dried over Na₂SO₄ and concentrated. The crude product was loaded onto silica gel and eluted with a gradient of ethyl acetate in hexanes going from 80% to 100% to afford the title compound as a white solid. ¹H NMR (400 MHz, d₆-acetone): δ 11.15 (bs, 1H), 7.68-7.61 (m, 2 H), 7.39 (d, 1H), 7.15-7.10 (m, 1H), 5.08-5.04 (m, 1H), 3.88-3.80 (m, 4H), 3.65 (s, 3H), 3.06-3.02 (t, 2H), 2.91-2.89 (t, 2H), 2.23-2.15 (m, 2H), 2.07-1.98 (m, 2H) ppm. MS (APCI, Q⁺) m/z 483.2 [M+H]⁺.

### Example 3

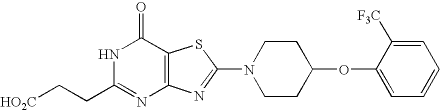

### 3-(7-Oxo-2-{4-[2-(trifluoromethyl)phenoxy]piperidin-1-yl}-6,7-dihydro[1,3]thiazolo[4,5-d]pyrimidin-5-yl)propanoic acid

To an ice-cold solution of methyl 3-(7-oxo-2-{4-[2-(trifluoromethyl)phenoxy]-piperidin-1-yl}-6,7-dihydro[1,3]thiazolo[4,5-d]pyrimidin-5-yl)propanoate (36 mg, 0.075 mmol, from Example 2) in THF (0.7 mL) and methanol (0.3 mL) was added dropwise 1.0 N aqueous lithium hydroxide (0.3 mL, 0.3 mmol). The resulting solution was stirred at rt for 4 h. The reaction mixture was acidified by addition of aqueous KH₂PO₄ and extracted with boiling EtOAc (2×20 mL). The organic layer was dried over Na₂SO₄ and concentrated to give a yellow solid. The crude product was triturated with Et₂O (8 mL) and collected by filtration to give the title compound as a white solid. ¹HNMR (400 MHz, d₆-acetone): δ 11.05 (bs, 1H), 7.68-7.64 (m, 2 H), 7.39 (d, 1H), 7.13 (t, 1H), 5.06 (m, 1H), 3.87-3.80 (m, 4H), 3.03 (t, 2H), 2.91 (t, 2H), 2.24-2.15 (m, 2H), 2.07-1.98 (t, 2H) ppm. MS (APCI, Q⁺) m/z 469.2 [M+H]⁺.

### Example 4

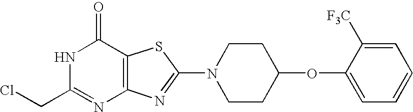

### 5-(Chloromethyl)-2-{4-[2-(trifluoromethyl)phenoxy]piperidin-1-yl}[1,3]thiazolo[4,5-d]pyrimidin-7(6H)-one

A mixture of 4-amino-2-{4-[2-(trifluoromethyl)phenoxy]piperidin-1-yl}-1,3-thiazole-5-carboxamide (0.40 g, 1.04 mmol, from Step 4 of Example 1) with chloroacetic acid (2.56 g) was warmed to 80° C. to give a homogeneous solution to which was slowly added chloroacetyl chloride (0.17 mL, 2.1 mmol). The resulting mixture was stirred at 130° C. for 5 h. The reaction was allowed to cool to rt and was partitioned between EtOAc and half-saturated NaHCO₃, dried over Na₂SO₄, and concentrated. The crude product was triturated and sonicated in EtOAc (7 mL), collected by filtration, and dried to give the title compound as a light beige solid. ¹H NMR (400 MHz, d₆-acetone): δ 11.42 (1H, br. s), 7.64 (2H, m), 7.39 (1H, d), 7.12 (1H, dd), 5.08 (1H, m), 4.62 (2H, s), 3.86 (4H, m), 2.20 (2H, m), 2.02 (2H, m) ppm. MS (ESI, Q⁺) 445.0 (M+1).

### Example 5

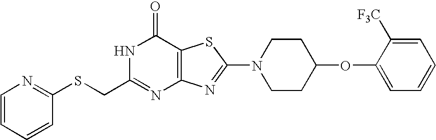

### 5-[(pyridin-2-ylthio)methyl]-2-{4-[2-(trifluoromethyl)phenoxy]piperidin-1-yl}[1,3]thiazolo[4,5-d]pyrimidin-7(6H)-one

To an ice-cold solution of 5-(chloromethyl)-2-{4-[2-(trifluoromethyl)phenoxy]-piperidin-1-yl}[1,3]thiazolo[4,5-d]pyrimidin-7(6H)-one (43 mg, 0.097 mmol, from Example 4) and 2-mercaptopyridine (21 mg, 0.19 mmol) in dichloromethane (1 mL) was added triethylamine (0.04 mL, 0.29 mmol). The resulting solution was stirred at rt for 0.5 h. The reaction mixture was partitioned between EtOAc and half-saturated NaHCO₃, the organic layer was dried over Na₂SO₄ and concentrated. The crude product was loaded onto silica gel and eluted with a gradient of ethyl acetate in hexanes going from 70% to 100% to give the title compound as a white solid. ¹H NMR (400 MHz, d₆-acetone): δ 11.9 (bs, 1H), 8.61 (ddd, 1H), 7.78-7.75 (m, 1 H), 7.68-7.62 (m, 2H), 7.48 (dt, 1H), 7.39 (d, 1H), 7.30-7.28 (m, 1H), 7.13 (t, 1H), 5.07-5.05 (m, 1H), 4.38 (s, 2H), 3.87-3.82 (m, 4H), 2.24-2.16 (m, 2H), 2.10-1.98 (m, 2H) ppm.

MS (ESI, Q⁺) m/z 520.2 [M+H]⁺.

### Example 6

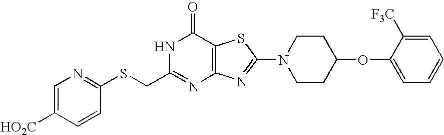

### 6-{[(7-oxo-2-{4-[2-(trifluoromethyl)phenoxy]piperidin-1-yl}-6,7-dihydro[1,3]thiazolo[4,5-d]pyrimidin-5-yl)methyl]thio}nicotinic acid

The title compound was prepared as described for Example 5, replacing the 2-mercaptopyridine by 6-mercaptonicotinic acid. MS (ESI, Q⁺) m/z 564.0 (M+1).

### Example 7

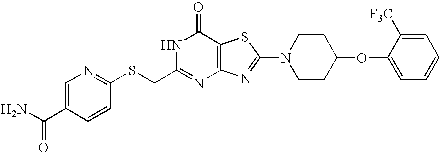

### 6-{[(7-oxo-2-{4-[2-(trifluoromethyl)phenoxy]piperidin-1-yl}-6,7-dihydro[1,3]thiazolo[4,5-d]pyrimidin-5-yl)methyl]thio}nicotinic acid

The title compound was prepared as described for Example 5, replacing the 2-mercaptopyridine by 6-mercaptonicotinamide. MS (ESI, Q⁺) m/z 563.1 (M+1).

### Example 8

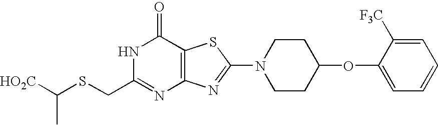

### 2-{[(7-oxo-2-{4-[2-(trifluoromethyl)phenoxy]piperidin-1-yl}-6,7-dihydro[1,3]thiazolo[4,5-d]pyrimidin-5-yl)methyl]thio}propanoic acid

The title compound was prepared as described for Example 5, replacing the 2-mercaptopyridine by methyl 2-mercaptopropanoate followed by saponification as described in Example 3. MS (ESI, Q⁺) m/z 515 [M+H]⁺.

### Example 9

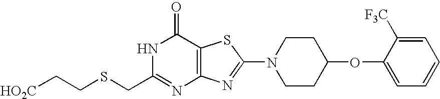

### 3-{[(7-oxo-2-{4-[2-(trifluoromethyl)phenoxy]piperidin-1-yl}-6,7-dihydro[1,3]thiazolo[4,5-d]pyrimidin-5-yl)methyl]thio}propanoic acid

The title compound was prepared as described for Example 5, replacing the 2-mercaptopyridine by methyl 3-mercaptopropanoate followed by saponification as described in Example 3. MS (ESI, Q⁺) m/z 515 [M+H]⁺.

### Example 10

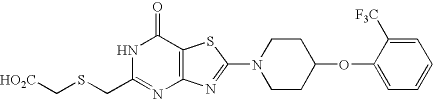

### {[(7-oxo-2-{4-[2-(trifluoromethyl)phenoxy]piperidin-1-yl}-6,7-dihydro[1,3]thiazolo[4,5-d]pyrimidin-5-yl)methyl]thio}acetic acid

The title compound was prepared as described for Example 5, replacing the 2-mercaptopyridine by methyl thioglycolate followed by saponification as described in Example 3.

MS (ESI, Q⁺) m/z 501 [M+H]⁺.

### Example 11

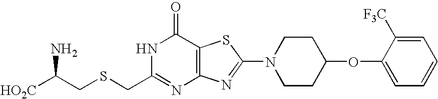

### S-[(7-oxo-2-{4-[2-(trifluoromethyl)phenoxy]piperidin-1-yl}-6,7-dihydro[1,3]thiazolo[4,5-d]pyrimidin-5-yl)methyl]-L-cysteine

The title compound was prepared as described for Example 5, replacing the 2-mercaptopyridine by methyl L-cysteinate followed by saponification as described in Example 3.

MS (ESI, Q⁺) m/z 530 [M+H]⁺.

### Example 12

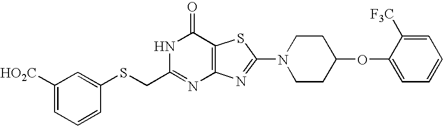

### 3-{[(7-oxo-2-{4-[2-(trifluoromethyl)phenoxy]piperidin-1-yl}-6,7-dihydro[1,3]thiazolo[4,5-d]pyrimidin-5-yl)methyl]thio}benzoic acid

The title compound was prepared as described for Example 5, replacing the 2-mercaptopyridine by 3-mercaptobenzoic acid followed by saponification as described in Example 3. MS (ESI, Q⁺) m/z 563 [M+H]⁺.

### Example 13

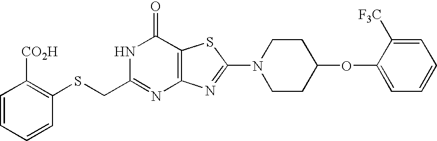

### 2-{[(7-oxo-2-{4-[2-(trifluoromethyl)phenoxy]piperidin-1-yl}-6,7-dihydro[1,3]thiazolo[4,5-d]pyrimidin-5-yl)methyl]thio}benzoic acid

The title compound was prepared as described for Example 5, replacing the 2-mercaptopyridine by 2-mercaptobenzoic acid followed by saponification as described in Example 3. MS (ESI, Q⁺) m/z 563 [M+H]⁺.

### Example 14

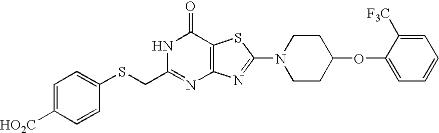

### 4-{[(7-oxo-2-{4-[2-(trifluoromethyl)phenoxy]piperidin-1-yl}-6,7-dihydro[1,3]thiazolo[4,5-d]pyrimidin-5-yl)methyl]thio}benzoic acid

The title compound was prepared as described for Example 5, replacing the 2-mercaptopyridine by 4-mercaptobenzoic acid followed by saponification as described in Example 3. MS (ESI, Q⁺) m/z 563 [M+H]⁺.

### Example 15

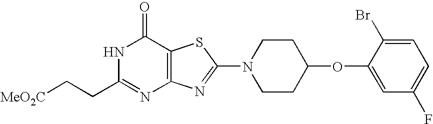

### Methyl 3-{2-[4-(2-bromo-5-fluorophenoxy)piperidin-1-yl]-7-oxo-6,7-dihydro[1,3]thiazolo[4,5-d]pyrimidin-5-yl}propanoate

### Step 1: Methyl N-cyano-4-hydroxypiperidine-1-carbimidothioate

4-Hydroxypiperidine (10.2 g, 101 mmol) and dimethyl N-cyanodithioiminocarbonate (14.7 g, 101 mmol) were heated together at 65° C. in ethanol (100 mL) for 2.5 h. The mixture was then concentrated in vacuo to afford the title compound as a red-orange solid.

### Step 2: 4-Amino-2-(4-hydroxypiperidin-1-yl)-1,3-thiazole-5-carboxamide

At room temperature, triethylamine (12.0 mL, 15 mmol) was slowly added to a mixture of methyl N-cyano-4-hydroxypiperidine-1-carbimidothioate (1.56 g, 4.57 mmol) and 2-mercaptoacetamide (4.2 mL, 4.6 mmol, 10 wt % in methanolic ammonia) and the resulting solution was stirred overnight. Water (5 mL) was added and stirring was continued for 1 h. The solid that was collected by filtration was washed with an ice cold solution of water/methanol (1:2). It was further dried under vacuum to afford the title compound as a light-orange powder.

### Step 3: 1-[5-(3-methoxy-3-oxopropyl)-7-oxo-6,7-dihydro[1,3]thiazolo[4,5-d]pyrimidin-2-yl]piperidin-4-yl methyl succinate

At room temperature, methyl 4-chloro-4-oxobutanoate (1.5 mL, 12.2 mmol) was added to a suspension of 4-amino-2-(4-hydroxypiperidin-1-yl)-1,3-thiazole-5-carboxamide (1.0 g, 4.1 mmol) in dimethyl succinate (6.9 mL). The resulting mixture was stirred at 120° C. for 2 h. It was cooled to rt, diluted with EtOAc, quickly washed with half-saturated NaHCO₃, dried over Na₂SO₄ and concentrated. The crude product was purified by chromatography (applied with DMSO) eluting with EtOH in EtOAc going from 0 to 10% to afford the title compound as a beige solid.

### Step 4: Methyl 3-[2-(4-hydroxypiperidin-1-yl)-7-oxo-6,7-dihydro[1,3]thiazolo[4,5-d]pyrimidin-5-yl]propanoate

To a suspension of 1-[5-(3-methoxy-3-oxopropyl)-7-oxo-6,7-dihydro[1,3]thiazolo-[4,5-d]pyrimidin-2-yl]piperidin-4-yl methyl succinate (0.55 g, 1.2 mmol) in methanol (6 mL) was added a solution of 0.94M sodium methoxide in methanol (1.15 mL, 1.4 mmol). The resulting solution was stirred at rt for 1 h. The reaction mixture was partitioned between EtOAc and aqueous NH₄OAc (25% w/v), the organic layer was dried over Na₂SO₄ and concentrated. The crude product was purified by chromatography (applied with DMSO) eluting with EtOH in EtOAc going from 0 to 20% to afford the title compound as a beige solid.

### Step 5: Methyl 3-{2-[4-(2-bromo-5-fluorophenoxy)piperidin-1-yl]-7-oxo-6,7-dihydro[1,3]thiazolo[4,5-d]pyrimidin-5-yl}propanoate

Diethyl azodicarboxylate (0.065 mL, 0.41 mmol) was added dropwise to an ice-cold suspension of methyl 3-[2-(4-hydroxypiperidin-1-yl)-7-oxo-6,7-dihydro[1,3]thiazolo-[4,5-d]pyrimidin-5-yl]propanoate (110 mg, 0.33 mmol), 2-bromo-5-fluorophenol (75 mg, 0.39 mmol) and triphenylphosphine (137 mg. 0.52 mmol) in THF (1 mL). The mixture was then warmed to rt and stirred for 3 d before being concentrated and partitioned between EtOAc and aqueous NH₄OAc (25% w/v). The organic layer was dried over Na₂SO₄ and concentrated. The crude product was purified by chromatography (applied with DMSO) eluting with EtOAc in hexane going from 80 to 100% to afford the title compound as a pale yellow solid. ¹H NMR (400 MHz, d₆-acetone): δ 11.13-11.08 (br s, 1H), 7.66-7.60 (m, 1H), 7.14 (dd, 1H), 6.79-6.73 (m, 1H), 5.03-4.97 (m, 1H), 3.95-3.76 (m, 4H), 3.65 (s, 3H), 3.03 (td, 2H), 2.90 (t, 2H), 2.23-2.14 (m, 2H), 2.08-1.98 (m, 2H) ppm. MS (ESI, Q⁺) m/z 510.9, 512.9 [M+H]⁺.

### Example 16

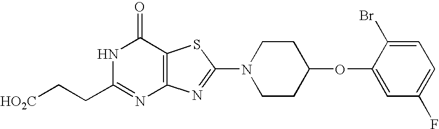

### 3-{2-[4-(2-bromo-5-fluorophenoxy)piperidin-1-yl]-7-oxo-6,7-dihydro[1,3]thiazolo-[4,5-d]pyrimidin-5-yl}propanoic acid

The title compound was prepared as described for Example 3, replacing the methyl 3-(7-oxo-2-{4-[2-(trifluoromethyl)phenoxy]piperidin-1-yl}-6,7-dihydro[1,3]thiazolo[4,5-d]pyrimidin-5-yl)propanoate by methyl 3-{2-[4-(2-bromo-5-fluorophenoxy)piperidin-1-yl]-7-oxo-6,7-dihydro[1,3]thiazolo[4,5-d]pyrimidin-5-yl}propanoate. ¹H NMR (400 MHz, d₆-acetone): δ 12.1-11.9 (br s, 1H), 7.63 (dd, 1H), 7.18 (dd, 1H), 6.77 (m, 1H), 5.01-4.99 (m, 1H), 3.93-3.78 (m, 4H), 2.95 (t, 2H), 2.81 (t, 2H), 2.21-2.13 (m, 2H), 2.03-1.95 (m, 2 H) ppm. MS (ESI, Q⁺) m/z 497, 499 [M+H]⁺.

### Example 17

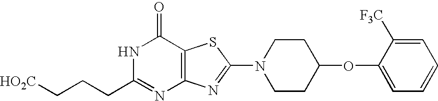

### 4-(7-oxo-2-{4-[2-(trifluoromethyl)phenoxy]piperidin-1-yl}-6,7-dihydro[1,3]thiazolo pyrimidin-5-yl)butanoic acid

The title compound was prepared as described for Example 2, replacing the carbomethoxypropionyl chloride by methyl 4-(chloroformyl)butyrate and the dimethyl succinate by dimethyl glutarate. The crude product was triturated with EtOAc followed by saponification as described in Example 3. MS (APCI, Q⁺) m/z 483 [M+H]⁺.

### Example 18

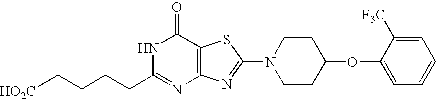

### 5-(7-oxo-2-{4-[2-(trifluoromethyl)phenoxy]piperidin-1-yl}-6,7-dihydro[1,3]thiazolo[4,5-d]-pyrimidin-5-yl)pentanoic acid

The title compound was prepared as described for Example 2, replacing the carbomethoxypropionyl chloride by methyl adipoyl chloride and the dimethyl succinate by dimethyl adipate. The crude product was purified by chromatography on silica gel eluting with EtOH in EtOAc going from 0 to 5% to afford the corresponding methyl ester which was hydrolysed to the acid as described in Example 3. MS (ESI, Q⁺) m/z 497 [M+H]⁺.

### Example 19

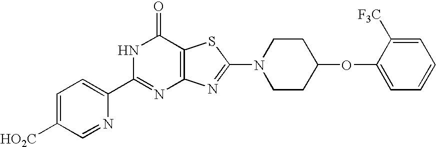

### 6-(7-Oxo-2-{4-[2-(trifluoromethyl)phenoxy]piperidin-1-yl}-6,7-dihydro[1,3]thiazolo-[4,5-d]pyrimidin-5-yl)nicotinic acid

### Step 1: Methyl 6-({[(4-amino-2-{4-[2-(trifluoromethyl)phenoxy]piperidin-1-yl}-1,3-thiazol-5-yl)carbonyl]amino}carbonyl)nicotinate

To a solution of 5-(methoxycarbonyl)pyridine-2-carboxylic acid (56 mg, 0.31 mmo) and oxalyl chloride (0.03 mL, 0.33 mmol) in toluene (2 mL) was added DMF (0.2 mL, 2.6 mmol). The reaction mixture was stirred at rt for 30 min and the solvents were removed under reduced pressure. A solution of 4-amino-2-{4-[2-(trifluoromethyl)phenoxy]piperidin-1-yl}-1,3-thiazole-5-carboxamide (100 mg, 0.26 mmol, from Step 4 of Example 1) in DMF was then added followed by sodium hydride (60% oil dispersion) (10 mg, 0.26 mmol) and the resulting mixture was stirred at rt for 3 d. The mixture was then partitioned between EtOAc and aqueous KH₂PO₄. The aqueous layer was extracted three times with EtOAc. The combined organic layers were dried over Na₂SO₄ and concentrated. The crude product was purified by chromatography on silica gel eluting with 50 to 100% EtOAc in hexane to afford the title compound.

### Step 2: Methyl 6-(7-oxo-2-{4-[2-(trifluoromethyl)phenoxy]piperidin-1-yl}-6,7-dihydro[1,3]thiazolo[4,5-d]pyrimidin-5-yl)nicotinate

A solution of methyl 6-({[(4-amino-2-{4-[2-(trifluoromethyl)phenoxy]piperidin-1-yl}-1,3-thiazol-5-yl)carbonyl]amino}carbonyl)nicotinate (15 mg, 0.03 mmol) and camphorsulfonic acid (6 mg, 0.03 mmol) in xylene was refluxed using a Dean-Stark trap for 2 h. The mixture was allowed to cool to rt and then aqueous NaHCO₃ was added. The aqueous layer was extracted twice with EtOAc and the combined organic layers were dried over Na₂SO₄ and concentrated. The crude product was purified by chromatography on silica gel eluting with EtOAc to afford the title compound.

### Step 3: 6-(7-Oxo-2-{4-[2-(trifluoromethyl)phenoxy]piperidin-1-yl}-6,7-dihydro[1,3]thiazolo[4,5-d]pyrimidin-5-yl)nicotinic acid

The title compound was obtained by hydrolysis of the methyl ester from Step 2 as described for Example 3, replacing the methyl 3-(7-oxo-2-{4-[2-(trifluoromethyl)phenoxy]piperidin-1-yl}-6,7-dihydro[1,3]thiazolo[4,5-d]pyrimidin-5-yl)propanoate by methyl 6-(7-oxo-2-{4-[2-(trifluoromethyl)phenoxy]piperidin-1-yl}-6,7-dihydro[1,3]thiazolo[4,5-d]pyrimidin-5-yl)nicotinate. ¹H NMR (400 MHz, d₆-acetone): δ 11.2-11.0 (br s, 1H), 9.27 (s, 1H), 8.66-8.59 (m, 2H), 7.70-7.62 (m, 2H), 7.41 (d, 1H), 7.14 (t, 1 H), 5.09 (m, 1H), 3.92-3.87 (m, 4H), 2.27-2.21 (m, 2H), 2.10-2.05 (m, 2H) ppm.

MS (ESI, Q⁺) m/z 518 [M+H]⁺.

### Example 20

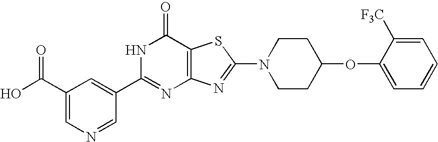

### 5-(7-Oxo-2-{4-[2-(trifluoromethyl)phenoxy]piperidin-1-yl}-6,7-dihydro[1,3]thiazolo-[4,5-d]pyrimidin-5-yl)nicotinic acid

### Step 1: 3-Carboxy-5-(ethoxycarbonyl)pyridinium chloride

To a refluxing solution of diethyl pyridine-3,5-dicarboxylate (10 g, 44.8 mmol) in ethanol (180 mL) and chloroform (24 mL) was added dropwise an aqueous solution of 1N KOH (44.8 mL, 44.8 mmol). After 30 min, it was allowed to cool to room temperature and poured onto 1 L of diethyl ether. The mixture was cooled in ice for 30 min and the precipitate was collected by filtration. The resulting solid was dissolved in a minimum of water and added to saturated aqueous KH₂PO₄. It then precipitated and the resulting white solid was collected by filtration. This solid was added to a 10% aq. HCl solution and collected by filtration again to afford the title compound.

### Step 2: 3-(Chlorocarbonyl)-5-(ethoxycarbonyl)pyridinium chloride

A solution of 3-carboxy-5-(ethoxycarbonyl)pyridinium chloride and thionyl chloride was heated to 80° C. for 3 h. The mixture was concentrated and the resulting acid chloride was used without purification in the next step.

### Step 3: 5-(7-Oxo-2-{4-[2-(trifluoromethyl)phenoxy]piperidin-1-yl}-6,7-dihydro[1,3]thiazolo[4,5-d]pyrimidin-5-yl)nicotinic acid

The title compound was prepared as described for Example 2, replacing the carbomethoxypropionyl chloride by 3-(chlorocarbonyl)-5-(ethoxycarbonyl)pyridinium chloride and the dimethyl succinate by 3-carboxy-5-(ethoxycarbonyl)pyridinium hydrochloride. The crude product was triturated with EtOAc followed by saponification as described in Example 3.

MS (ESI, Q⁺) m/z 518 [M+H]⁺.

### Example 21

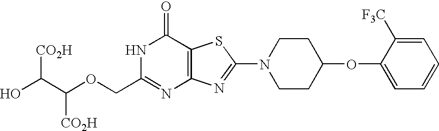

### 2-Hydroxy-3-[(7-oxo-2-{4-[2-(trifluoromethyl)phenoxy]piperidin-1-yl}-6,7-dihydro[1,3]thiazolo[4,5-d]pyrimidin-5-yl)methoxy]succinic acid

To a solution of dimethyl tartrate (12 mg, 0.67 mmol, 1:1 mixture of D and L) and 5-(chloromethyl)-2-{4-[2-(trifluoromethyl)phenoxy]piperidin-1-yl}[1,3]thiazolo[4,5-d]pyrimidin-7(6H)-one (15 mg, 0.034 mmol, from Example 4) in DMF (750 μL, 0.045M) was added sodium hydride (4 mg of 60% oil dispersion, 0.10 mmol) and the mixture was stirred 2 d at rt. The reaction mixture containing the corresponding methyl ester was hydrolysed to the acid as described in Example 3. Formic acid was added to quench the reaction. Volatile components were removed under vacuum and the solution was reconstituted in 1 mL of DMSO. The product was purified using semi-preparative LC-MS. MS (ESI, Q⁺) m/z 559 [M+H]⁺.

### Example 22

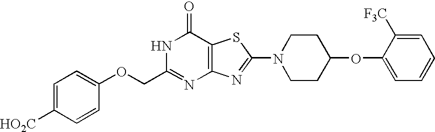

### 4-[(7-Oxo-2-{4-[2-(trifluoromethyl)phenoxy]piperidin-1-yl}-6,7-dihydro[1,3]thiazolo[4,5-d]pyrimidin-5-yl)methoxy]benzoic acid

To a solution of the methyl 4-hydroxybenzoate (11 mg, 0.075 mmol) and 5-(chloromethyl)-2-{4-[2-(trifluoromethyl)phenoxy]piperidin-1-yl}[1,3]thiazolo[4,5-d]pyrimidin-7(6H)-one (15 mg, 0.034 mmol, from Example 4) in triglyme (750 μL) was added potassium carbonate (20 mg, 0.14 mmol) and the suspension was stirred rt for 2 d. The reaction mixture containing the methyl ester was hydrolysed as described in Example 3. Formic acid was added to quench the reaction. The product was purified using semi-preparative LC-MS. MS (ESI, Q⁺) m/z 547 [M+H]⁺.

### Example 23

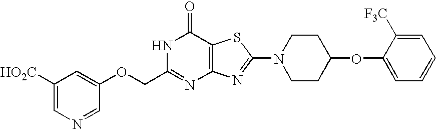

### 5-[(7-Oxo-2-{4-[2-trifluoromethyl)phenoxy]piperidin-1-yl}-6,7-dihydro[1,3]thiazolo[4,5-d]pyrimidin-5-yl)methoxy]nicotinic acid

The title compound was prepared as described for Example 22, replacing the methyl 4-hydroxybenzoate by methyl 5-hydroxynicotinate. MS (ESI, Q⁺) m/z 548 [M+H]⁺.

### Example 24

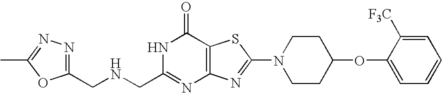

### 5-({[(5-Methyl-1,3,4-oxadiazol-2-yl)methyl]amino}methyl)-2-{4-[2-(trifluoromethyl)phenoxy]piperidin-1-yl}[1,3]thiazolo[4,5-d]pyrimidin-7(6H)-one

To a solution of (5-methyl-1,3,4-oxadiazol-2-yl)methanaminium oxalate (14 mg, 0.69 mmol) and 5-(chloromethyl)-2-{4-[2-(trifluoromethyl)phenoxy]piperidin-1-yl}[1,3]thiazolo[4,5-d]pyrimidin-7(6H)-one (15 mg, 0.034 mmol, from Example 4) in DMF (1 mL) was added triethylamine (23 μL, 0.165 mmol) and the solution was stirred overnight at rt. Acetic acid was added to quench the reaction. Volatile components were removed under vacuum and the solution was reconstituted in 1 mL of DMSO. The product was purified using semi-preparative LC-MS.

MS (ESI, Q⁺) m/z 522.1 [M+H]⁺.

### Example 25

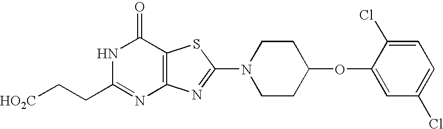

### 3-{2-[4-(2,5-Dichlorophenoxy)piperidin-1-yl]-7-oxo-6,7-dihydro[1,3]thiazolo[4,5-d]pyrimidin-5-yl}propanoic acid

The title compound was prepared as described for Example 15, replacing in step 5 the 2-bromo-5-fluorophenol with 2,5-dichlorophenol to afford the corresponding methyl ester which was hydrolysed as described in Example 3. MS (ESI, Q⁻) m/z 467, 469 [M−H]⁻.

### Example 26

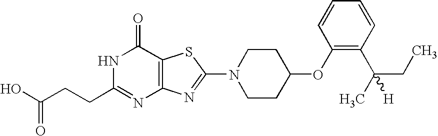

### 3-{2-[4-(2-sec-Butylphenoxy)piperidin-1-yl]-7-oxo-6,7-dihydro[1,3]thiazolo[4,5-d]pyrimidin-5-yl}propanoic acid

The title compound was prepared as described for Example 15, replacing in step 5 the 2-bromo-5-fluorophenol with 2-sec-butylphenol to afford the corresponding methyl ester which was hydrolysed as described in Example 3. MS (ESI, Q⁺) m/z 457 [M+1].

### Example 27

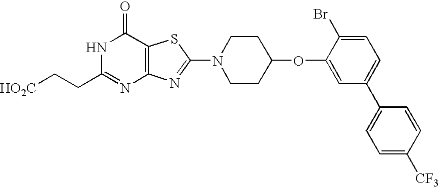

### 3-[2-(4-{[4-Bromo-4′-(trifluoromethyl)biphenyl-3-yl]oxy}piperidin-1-yl)-7-oxo-6,7-dihydro[1,3]thiazolo[4,5-d]pyrimidin-5-yl]propanoic acid

The title compound was prepared as described for Example 15, replacing in step 5 the 2-bromo-5-fluorophenol with 4-bromo-4′-(trifluoromethyl)biphenyl-3-ol to afford the corresponding methyl ester which was hydrolysed as described in Example 3. MS (ESI, Q⁻) m/z 543, 545 [M−H]⁻.

### Example 28

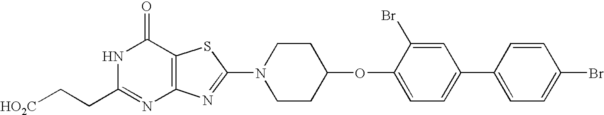

### 3-(2-{4-[(3,4′-dibromobiphenyl-4-yl)oxy]piperidin-1-yl}-7-oxo-6,7-dihydro[1,3]thiazolo[4,5-d]pyrimidin-5-yl)propanoic acid

The title compound was prepared as described for Example 15, replacing in step 5 the 2-bromo-5-fluorophenol with 3,4′-dibromobiphenyl-4-ol to afford the corresponding methyl ester which was hydrolysed as described in Example 3. MS (APCI, Q⁺) m/z 615, 617, 619 [MH−H₂O]⁺.

### Example 29

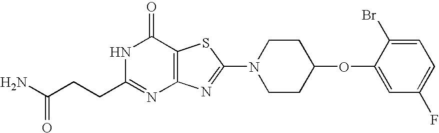

### 3-{2-[4-(2-bromo-5-fluorophenoxy)piperidin-1-yl]-7-oxo-6,7-dihydro[1,3]thiazolo[4,5-d]pyrimidin-5-yl}propanamide

To an ice-cold solution of 3-{2-[4-(2-bromo-5-fluorophenoxy)piperidin-1-yl]-7-oxo-6,7-dihydro[1,3]thiazolo-[4,5-d]pyrimidin-5-yl}propanoic acid from Example 16 (61 mg, 0.12 mmol) and HATU (380 mg, 0.24 mmol) in DMF (6 mL) was added ammonium hydroxide (0.05 mL, 0.7 mmol). The resulting yellow solution was stirred at room temperature for 4 h. The mixture was partitioned between EtOAc and NH₄OAc, the organic layer was dried over Na₂SO₄ and concentrated. The resulting residue was purified by flash chromatography on silica gel (applied using a minimum of DMSO) eluted with a gradient of conc. NH₄OH/EtOH/CHCl₃ progressing from (0:0:100) to (0:20:80) and then to (1:20:79) to afford the title compound as a white solid. MS (ESI, Q⁻) m/z 494, 496 [M−H]⁻.

### Example 30

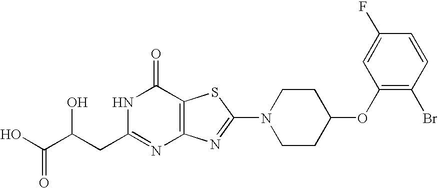

### 3-{2-[4-(2-Bromo-5-fluorophenoxy)piperidin-1-yl]-7-oxo-6,7-dihydro[1,3]thiazolo[4,5-d]pyrimidin-5-yl}-2-hydroxypropanoic acid

### Step 1: Methyl 3-[2-[4-(2-bromo-5-fluorophenoxy)piperidin-1-yl]-6-methoxymethyl)-7-oxo-6,7-dihydro[1,3]thiazolo[4,5-d]pyrimidin-5-yl]propanoate

Into a 100 mL flask equipped with a magnetic stirbar was added sodium hydride (156 mg, 3.90 mmol) and THF (10.0 mL). The suspension was treated with bromomethyl methyl ether (510 μL, 6.26 mmol) and cooled to 0° C. To this reaction mixture was added methyl 3-{2-[4-(2-bromo-5-fluorophenoxy)piperidin-1-yl]-7-oxo-6,7-dihydro[1,3]thiazolo[4,5-d]pyrimidin-5-yl}propanoate (800 mg, 1.56 mmol, dissolved in 10 mL of THF, and 1 mL of DMF) dropwise over 15 min. The resulting suspension was stirred at 0° C. for 30 min and then at 25° C. for 30 min. The reaction mixture was quenched by dropwise addition of a saturated aqueous NH₄Cl solution (5 mL) and then poured into a 250 mL separatory funnel containing saturated aqueous NH₄Cl (100 mL) and the mixture was extracted with ethyl acetate (3×75 mL). The combined organic layers were washed with brine, dried over MgSO₄, filtered and the solvent was evaporated under reduced pressure. Purification by column chromatography through silica gel, eluting with 50% EtOAc in hexanes to 100% EtOAc in hexanes as a gradient, gave the title compound as a light yellow foam. MS (ESI, Q⁺) m/z 555, 557 [M+1].

### Step 2: Methyl 3-[2-[4-(2-bromo-5-fluorophenoxy)piperidin-1-yl]-6-(methoxymethyl)-7-oxo-6,7-dihydro[1,3]thiazolo[4,5-d]pyrimidin-5-yl]-2-hydroxypropanoate

Into a flame-dried 25 mL round-bottom flask equipped with a magnetic stirbar and under N₂ was added methyl 3-[2-[4-(2-bromo-5-fluorophenoxy)piperidin-1-yl]-6-(methoxymethyl)-7-oxo-6,7-dihydro[1,3]thiazolo[4,5-d]pyrimidin-5-yl]propanoate (100 mg, 0.18 mmol) and THF (2.0 mL). The solution was cooled to −78° C. and treated with 0.5 M potassium hexamethyldisilazide (0.72 ml, 0.36 mmol) in toluene. The resulting yellow solution was stirred at −78° C. for 30 min and then 3-phenyl-2-(phenylsulfonyl)oxaziridine (165 mg, 0.63 mmol) in 1 mL of THF was added in a single addition. The reaction mixture was stirred at −78° C. for 1 h and then quenched by dropwise addition of a saturated aqueous NH₄Cl solution (5 mL) with warming to room temperature. The mixture was poured into a 125 mL separatory funnel containing saturated aqueous NH₄Cl (75 mL) and extracted with ethyl acetate (3×30 mL). The combined organic layers were washed with brine, dried over MgSO₄, filtered and the solvent was evaporated under reduced pressure. Purification by column chromatography through silica gel, eluting with 40% EtOAc in hexanes to 80% EtOAc in hexanes as a gradient gave the title compound as a clear oil.

### Step 3: 3-{2-[4-(2-Bromo-5-fluorophenoxy)piperidin-1-yl]-7-oxo-6,7-dihydro[1,3]thiazolo[4,5-d]pyrimidin-5-yl}-2-hydroxypropanoic acid

Into a 5 mL flask equipped with a magnetic stirbar was added methyl 3-[2-[4-(2-bromo-5-fluorophenoxy)piperidin-1-yl]-6-(methoxymethyl)-7-oxo-6,7-dihydro[1,3]thiazolo[4,5-d]pyrimidin-5-yl]-2-hydroxypropanoate (40 mg, 0.07 mmol) and dichloromethane (1 mL). The reaction mixture was cooled to −78° C. and then 1.0 M boron tribromide (0.11 ml, 0.11 mmol) in dichloromethane was added in a single addition. The reaction was warmed to −40° C. and stirred for 1 h. The reaction mixture was quenched with dropwise addition of a 1M aqueous NaOH solution (1 mL). The reaction was warmed to room temperature and stirred for 2 h. The mixture was cooled, poured into a 125 mL separatory funnel containing pH 5 buffer (KH₂PO₄, 50 mL) and the mixture was extracted with ethyl acetate (3×30 mL). The combined organic layers were washed with brine, dried over MgSO₄, filtered and the solvent was evaporated under reduced pressure. Purification by column chromatography through silica gel, eluting with 100% CH₂Cl₂+0.5% AcOH to 95:5 CH₂Cl₂:MeOH+0.5% AcOH gave the desired compound as a white solid.

MS (ESI, Q⁺) m/z 513, 515 [M+1].

### Example 31

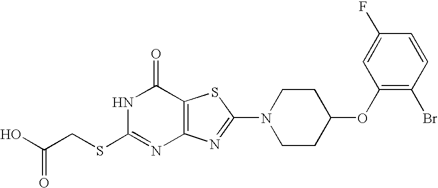

### ({2-[4-(2-Bromo-5-fluorophenoxy)piperidin-1-yl]-7-oxo-6,7-dihydro[1,3]thiazolo[4,5-d]pyrimidin-5-yl}thio)acetic acid

### Step 1: 2-[4-(2-bromo-5-fluorophenoxy)piperidin-1-yl]-5-mercapto[1,3]thiazolo[4,5-d]pyrimidin-7(6H-one)

Into a 250 mL flask equipped with a magnetic stirbar was added 4-amino-2-[4-(2-bromo 5-fluorophenoxy)piperidin-1-yl]-1,3-thiazole-5-carboxamide (2.00 g, 4.80 mmol), potassium ethylxanthate (0.990 mL, 9.6 mmol) and DMF (100 mL). The resulting suspension was heated to 100° C. for 2 h, and the reaction mixture was cooled and concentrated to remove the DMF. The crude reaction mixture was taken up in diethyl ether (100 mL), poured into a 250 mL separatory funnel containing pH 5 buffer (KH₂PO₄, 100 mL) and the mixture was extracted with diethyl ether (3×75 mL). The combined organic layers were washed with brine, dried over MgSO₄, filtered and the solvent was evaporated under reduced pressure. Purification by column chromatography through silica gel, eluting with 40% EtOAc in hexanes to 80% EtOAc in hexanes. The resulting brown foam obtained from concentration of the desired fractions was further purified by crystallization from hot dichloromethane and hexanes. The solid was filtered through Whatman #1 paper on a Hirsch funnel to give a yellow-orange solid.

MS (ESI, Q⁺) m/z 455, 457 [M+1].

### Step 2: Ethyl ({2-[4-(2-bromo-5-fluorophenoxy)piperidin-1-yl]-7-oxo-6,7-dihydro[1,3]thiazolo[4,5-d]pyrimidin-5-yl}thio)acetate

Into a 25 mL flask equipped with a magnetic stirbar was added 2-[4-(2-bromo-5-fluorophenoxy)piperidin-1-yl]-5-mercapto[1,3]thiazolo[4,5-d]pyrimidin-7(6H-one) (500 mg, 1.09 mmol), potassium carbonate (150 mg, 1.09 mmol) and DMF (2 mL). The resulting yellow solution was treated with dropwise addition of ethyl bromoacetate (0.120 ml, 1.09 mmol). The resulting solution was stirred at room temperature for 16 h. The mixture was poured into a 250 mL separatory funnel containing water (75 mL) and extracted with ethyl acetate (4×50 mL). The combined organic layers were washed with brine, dried over MgSO₄, filtered and the solvent was evaporated under reduced pressure. Purification by column chromatography through silica gel, eluting with 40% EtOAc in hexanes to 90% EtOAc in hexanes as a gradient gave the title compound as an orange solid. MS (ESI, Q⁺) m/z 543, 545 [M+1].

### Step 3: ({2-[4-(2-Bromo-5-fluorophenoxy)piperidin-1-yl]-7-oxo-6,7-dihydro[1,3]thiazolo[4,5-d]pyrimidin-5-yl}thio)acetic acid

Into a 25 mL flask equipped with a magnetic stirbar was added ethyl ({2-[4-(2-bromo-5-fluorophenoxy)piperidin-1-yl]-7-oxo-6,7-dihydro[1,3]thiazolo[4,5-d]pyrimidin-5-yl}thio)acetate (79 mg, 0.15 mmol), methanol (2.0 ml) and 1M aqueous lithium hydroxide (0.73 ml, 0.73 mmol). The resulting suspension was heated to 85° C. for 2 h. The reaction mixture was concentrated and the resulting suspension was poured into a 125 mL separatory funnel containing pH 5 buffer (KH₂PO₄, 50 mL) and the mixture was extracted with ethyl acetate (3×30 mL). The combined organic layers were washed with brine, dried over MgSO₄, filtered and the solvent was evaporated under reduced pressure to give the title compound as a beige solid.

MS (ESI, Q⁺) m/z 515, 517 [M+1].

### Example 32

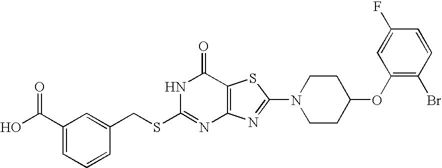

### 3-[({2-[4-(2-Bromo-5-fluorophenoxy)piperidin-1-yl]-7-oxo-6,7-dihydro[1,3]thiazolo-[4,5-d]pyrimidin-5-yl}thio)methyl]benzoic acid

The title compound was prepared as described for Example 31, replacing the ethyl bromoacetate by methyl 3-(bromomethyl)benzoate in Step 2. MS (APCI, Q⁻) m/z 589, 591 [M−1].

### Example 33

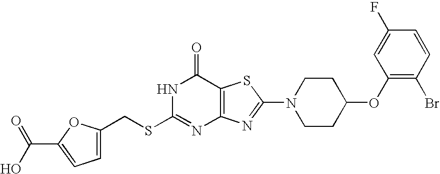

### 5-[({2-[4-(2-Bromo-5-fluorophenoxy)piperidin-1-yl]-7-oxo-6,7-dihydro[1,3]thiazolo[4,5-d]pyrimidin-5-yl}thio)methyl]-2-furoic acid

The title compound was prepared as described for Example 31, replacing the ethyl bromoacetate by methyl 5-(chloromethyl)-2-furoate in Step 2. MS (APCI, Q⁻) m/z 578, 581 [M−1].

### Example 34

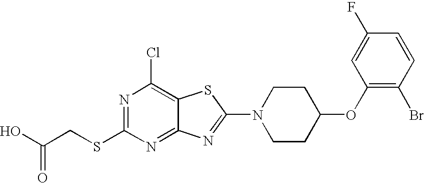

### ({2-[4-(2-Bromo-5-fluorophenoxy)piperidin-1-yl]-7-chloro[1,3]thiazolo[4,5-d]pyrimidin-5-yl}thio)acetic acid

### Step 1: Ethyl({2-[4-(2-bromo-5-fluorophenoxy)piperidin-1-yl]-7-chloro[1,3]thiazolo[4,5-d]pyrimidin-5-yl}thio)acetate

Into a 50 mL flask equipped with a magnetic stirbar was added ethyl({2-[4-(2-bromo-5-fluorophenoxy)piperidin-1-yl]-7-oxo-6,7-dihydro[1,3]thiazolo[4,5-d]pyrimidin-5-yl}thio)acetate (300 mg, 0.55 mmol) and DMF (42.7 μl, 0.55 mmol) in dichloromethane (10 mL). The brown solution was treated by dropwise addition of oxalyl chloride (480 μL, 5.5 mmol) and the brown solution was heated to reflux for 2 h. The cooled reaction mixture was concentrated under vacuum to remove the oxalyl chloride and dichloromethane. The residue was dissolved in ethyl acetate and poured into a 125 mL separatory funnel containing saturated aqueous NaHCO₃ (75 mL) and the mixture was extracted with ethyl acetate (3×30 mL). The combined organic layers were washed with brine, dried over MgSO₄, filtered and the solvent was evaporated under reduced pressure. Purification by flash chromatography through silica gel, eluting with 10% EtOAc in hexanes to 40% EtOAc in hexanes gave the title compound as a white foam. MS (ESI, Q⁺) m/z 563, 565 [M+1].

### Step 2: ({2-[4-(2-Bromo-5-fluorophenoxy)piperidin-1-yl]-7-chloro[1,3]thiazolo[4,5-d]pyrimidin-5-yl}thio)acetic acid

Into a 10 mL round-bottom flask equipped with a magnetic stirbar was added ethyl ({2-[4-(2-bromo-5-fluorophenoxy)piperidin-1-yl]-7-chloro[1,3]thiazolo[4,5-d]pyrimidin-5-yl}thio)acetate (70 mg, 0.13 mmol) in tetrahydrofuran (2 mL). The solution was treated with 1M aqueous lithium hydroxide (0.64 ml, 0.64 mmol) and stirred at room temperature for 4 h. The reaction mixture was concentrated and the crude mixture was poured into a 125 mL separatory funnel containing pH 5 buffer (KH₂PO₄, 50 mL) and the mixture was extracted with ethyl acetate (3×30 mL). The combined organic layers were washed with brine, dried over MgSO₄, filtered and the solvent was evaporated under reduced pressure. Purification by column chromatography through silica gel, eluting with 20:80 hexanes/EtOAc+1% AcOH gave the desired product as a white solid. MS (ESI, Q⁺) m/z 533, 535 [M+1].

### Example 35

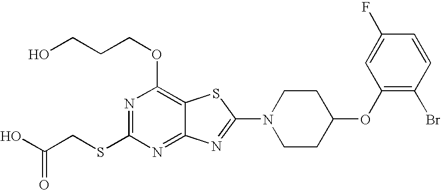

### {[2-[4-(2-Bromo-5-fluorophenoxy)piperidin-1-yl]-7-(3-hydroxypropoxy)[1,3]thiazolo[4,5-d]pyrimidin-5-yl]thio}acetic acid

Into a 25 mL round-bottom flask equipped with a magnetic stirbar was added ({2-[4-(2-bromo-5-fluorophenoxy)piperidin-1-yl]-7-chloro[1,3]thiazolo[4,5-d]pyrimidin-5-yl}thio)acetic acid (100 mg, 0.18 mmol), THF (2 ml) and 1,3-propanediol (135 mg, 1.80 mmol). The solution was treated with 1M aqueous sodium hydroxide (0.89 mL, 0.90 mmol) and refluxed for 2 h. The mixture was cooled, poured into a 125 mL separatory funnel containing pH 5 buffer (KH₂PO₄, 75 mL) and the mixture was extracted with ethyl acetate (3×30 mL). The combined organic layers were washed with brine, dried over MgSO₄, filtered and the solvent was evaporated under reduced pressure and purified by preparative HPLC through a C18 reverse phase column. MS (ESI, Q⁺) m/z 573, 575 [M+1].

### Example 36

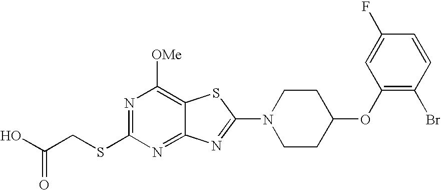

### {[2-[4-(2-Bromo-5-fluorophenoxy)piperidin-1-yl]-7-methoxy[1,3]thiazolo[4,5-d]pyrimidin-5-yl]thio}acetic acid

This compound was synthesized in a similar manner to Example 35, from ({2-[4-(2-bromo-5-fluorophenoxy)piperidin-1-yl]-7-chloro[1,3]thiazolo[4,5-d]pyrimidin-5-yl}thio)acetic acid and using methanol as a solvent. MS (ESI, Q⁺) m/z 529, 531 [M+1].

### Example 37

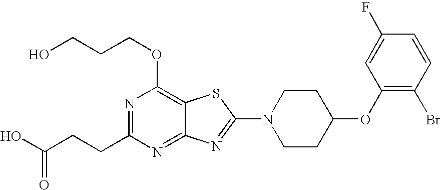

### 3-[2-[4-(2-Bromo-5-fluorophenoxy)piperidin-1-yl]-7-(3-hydroxypropoxy)[1,3]thiazolo-[4,5-d]pyrimidin-5-yl]propanoic acid

### Step 1: Methyl 3-{2-[4-(2-bromo-5-fluorophenoxy)piperidin-1-yl]-7-chloro[1,3]thiazolo[4,5-d]pyrimidin-5-yl}propanoate

Into a 50 mL flask equipped with a magnetic stirbar was added methyl 3-{2-[4-(2-bromo-5-fluorophenoxy)piperidin-1-yl]-7-oxo-6,7-dihydro[1,3]thiazolo[4,5-d]pyrimidin-5-yl}propanoate (500 mg, 0.98 mmol) and DMF (0.09 ml, 0.98 mmol) in CH₂Cl₂ (10 ml). The brown solution was treated by dropwise addition of oxalyl chloride (0.43 mL, 4.89 mmol) and refluxed for 2 h. The reaction mixture was concentrated to remove the oxalyl chloride and dichloromethane and the dark residue was dissolved in ethyl acetate and poured into a 125 mL separatory funnel containing 1M aqueous NaOH (75 mL) and the mixture was extracted with ethyl acetate (3×50 mL) The combined organic layers were washed with brine, dried over MgSO₄, filtered and the solvent was evaporated under reduced pressure. Purification by column chromatography through silica gel, eluting with 10% EtOAc in hexanes to 40% EtOAc in hexanes as a gradient gave the title compound as a light yellow oil.

### Step 2: 3-[2-[4-(2-Bromo-5-fluorophenoxy)piperidin-1-yl]-7-(3-hydroxypropoxy)[1,3]thiazolo[4,5-d]pyrimidin-5-yl]propanoic acid

Into a 25 mL round-bottom flask equipped with a magnetic stirbar was added methyl 3-{2-[4-(2-bromo-5-fluorophenoxy)piperidin-1-yl]-7-chloro[1,3]thiazolo[4,5-d]pyrimidin-5-yl}propanoate (60 mg, 0.11 mmol), 1,3-propanediol (86 mg, 1.13 mmol) and tetrahydrofuran (3.0 mL). The resulting solution was treated with 1M aqueous sodium hydroxide (0.57 mL, 0.57 mmol) and heated to reflux for 2 h. The mixture was cooled, poured into a 125 mL separatory funnel containing pH 5 buffer (KH₂PO₄, 75 mL) and the mixture was extracted with ethyl acetate (3×30 mL). The combined organic layers were washed with brine, dried over MgSO₄, filtered and the solvent was evaporated under reduced pressure to yield the title compound as a white solid. MS (ESI, Q⁺) m/z 555, 557 [M+1].

### Example 38

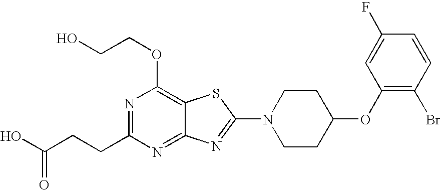

### 3-[2-[4-(2-Bromo-5-fluorophenoxy)piperidin-1-yl]-7-(2-hydroxyethoxy)[1,3]thiazolo[4,5-d]pyrimidin-5-yl]propanoic acid

This compound was synthesized in a similar manner to Example 37, from methyl 3-{2-[4-(2-bromo-5-fluorophenoxy)piperidin-1-yl]-7-chloro[1,3]thiazolo[4,5-d]pyrimidin-5-yl}propanoate (50 mg, 0.09 mmol) and ethylene glycol (230 mg, 4.7 mmol).

MS (ESI, Q⁺) m/z 541, 543 [M+1].

### Example 39

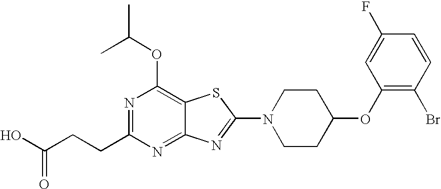

### 3-[2-[4-(2-Bromo-5-fluorophenoxy)piperidin-1-yl]-7-isopropoxy[1,3]thiazolo[4,5-d]pyrimidin-5-yl]propanoic acid

This compound was synthesized in a similar manner to Example 37, from methyl 3-{2-[4-(2-bromo-5-fluorophenoxy)piperidin-1-yl]-7-chloro[1,3]thiazolo[4,5-d]pyrimidin-5-yl}propanoate (50 mg, 0.09 mmol) and 2-propanol (2.0 mL).

MS (ESI, Q⁺) m/z 539, 541 [M+1].

### Example 40

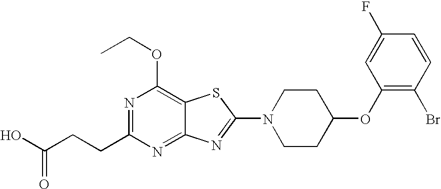

### 3-[2-[4-(2-Bromo-5-fluorophenoxy)piperidin-1-yl]-7-ethoxy[1,3]thiazolo[4,5-d]pyrimidin-5-yl]propanoic acid

This compound was synthesized in a similar manner to Example 37, from methyl 3-{2-[4-(2-bromo-5-fluorophenoxy)piperidin-1-yl]-7-chloro[1,3]thiazolo[4,5-d]pyrimidin-5-yl}propanoate (50 mg, 0.09 mmol) and ethanol (2.0 mL). MS (ESI, Q⁺) m/z 525, 527 [M+1].

### Example 41

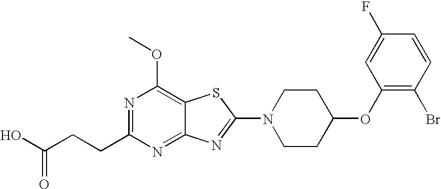

### 3-[2-[4-(2-Bromo-5-fluorophenoxy)piperidin-1-yl]-7-methoxy[1,3]thiazolo[4,5-d]pyrimidin-5-yl]propanoic acid

This compound was synthesized in a similar manner to Example 37, from methyl 3-{2-[4-(2-bromo-5-fluorophenoxy)piperidin-1-yl]-7-chloro[1,3]thiazolo[4,5-d]pyrimidin-5-yl}propanoate (50 mg, 0.09 mmol) and methanol (2.0 mL). MS (ESI, Q⁺) m/z 511, 513 [M+1].

### Example 42

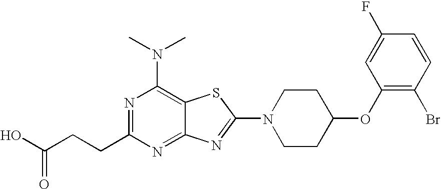

### 3-[2-[4-(2-Bromo-5-fluorophenoxy)piperidin-1-yl]-7-(dimethylamino)[1,3]thiazolo-[4,5-d]pyrimidin-5-yl]propanoic acid

### Step 1: Methyl 3-[2-[4-(2-Bromo-5-fluorophenoxy)piperidin-1-yl]-7-(dimethylamino)[1,3]thiazolo[4,5-d]pyrimidin-5-yl]propanoate

Into a 5 mL sealable flask equipped with a magnetic stirbar and under N₂ was added methyl 3-{2-[4-(2-bromo-5-fluorophenoxy)piperidin-1-yl]-7-chloro[1,3]thiazolo[4,5-d]pyrimidin-5-yl}propanoate (100 mg, 0.19 mmol) and 2.0 M dimethylamine (950 μL, 1.90 mmol) in THF. The resulting light orange solution was heated in an oil bath to 80° C. for 15 h. The reaction mixture was concentrated and purified by column chromatography through silica gel, eluting with 30% EtOAc in hexanes to 80% EtOAc in hexanes as a gradient, giving the title compound as a clear oil. MS (ESI, Q⁺) m/z 538, 540 [M+1].

### Step 2: 3-[2-[4-(2-Bromo-5-fluorophenoxy)piperidin-1-yl]-7-(dimethylamino)[1,3]thiazolo[4,5-d]pyrimidin-5-yl]propanoic acid

Into a 25 mL round-bottom flask equipped with a magnetic stirbar was added methyl 3-[2-[4-(2-bromo-5-fluorophenoxy)piperidin-1-yl]-7-(dimethylamino)[1,3]thiazolo-[4,5-d]pyrimidin-5-yl]propanoate (75 mg, 0.14 mmol), tetrahydrofuran (2.0 mL) and methanol (1.0 mL). The solution was treated with 1M aqueous lithium hydroxide (0.7 mL, 0.7 mmol) and stirred at room temperature for 2 h. The reaction mixture was concentrated, and poured into a 125 mL separatory funnel containing pH 5 buffer (KH₂PO₄, 50 mL) and the mixture was extracted with ethyl acetate (3×30 mL). The combined organic layers were washed with brine, dried over MgSO₄, filtered and the solvent was evaporated under reduced pressure. The desired product was isolated as a white foam. MS (ESI, Q⁺) m/z 524, 526 [M+1].

### Example 43

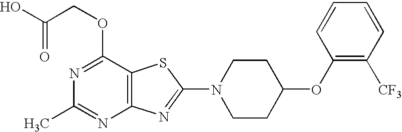

### ({2-[4-(2-Trifluoromethylphenoxy)piperidin-1-yl]-5-methyl[1,3]thiazolo[4-5,d]pyrimidin-7-yl}oxy)acetic acid

### Step 1: Methyl-2-{4-[2-(trifluoromethyl)phenoxy]piperidin-1-yl}[1,3]thiazolo[4,5-d]pyrimidin-7(6H)-one

To a mixture of 4-amino-2-{4-[2-(trifluoromethyl)phenoxy]piperidin-1-yl}-1,3-thiazole-5-carboxamide (0.40 g, 1.04 mmol, from Step 4 of Example 1) in acetic acid (2.50 g) was slowly added acetyl chloride (0.2 mL). The resulting mixture was stirred at 130° C. for 5 h. The reaction was allowed to cool to 25° C. and was partitioned between EtOAc and half-saturated aqueous NaHCO₃, dried over Na₂SO₄, and concentrated. The crude product was triturated and sonicated in EtOAc (7 mL), collected by filtration, and dried to give the title compound as a light beige solid.

### Step 2: 2-[4-(2-Trifluoromethylphenoxy)piperidin-1-yl]-7-chloro-5-methyl[1,3]thiazolo[4,5-d]pyrimidine

A 10 mL round-bottom flask containing a magnetic stirbar was charged with diethylaniline (195 μL, 1.22 mmol) and phosphorus oxychloride (3.7 mL, 40 mmol). The reaction mixture was stirred at room temperature for 10 min, and then 5-methyl-2-{4-[2-(trifluoromethyl)phenoxy]piperidin-1-yl}[1,3]thiazolo[4,5-d]pyrimidin-7(6H)-one (500 mg, 1.22 mmol) was added and the mixture was heated to 120° C. for 20 min. The reaction mixture was concentrated and the residue poured into a 125 mL separatory funnel containing 1M aqueous HCl (75 mL) and the mixture was extracted with ethyl acetate (3×30 mL). The combined organic layers were washed with brine, dried over MgSO₄, filtered and the solvent was evaporated under reduced pressure. Purification by column chromatography through silica gel, eluting with 50% EtOAc in hexanes to 100% EtOAc as a gradient afforded the title compound.

### Step 3: Ethyl({2-[4-(2-Trifluoromethylphenoxy)piperidin-1-yl]-5-methyl[1,3]thiazolo[4-5,d]pyrimidin-7-yl}oxy)acetate

A 10 mL round-bottom flask containing a magnetic stirbar containing ethyl glycolate (127 mg, 1.22 mmol) in toluene (1.0 mL) was treated with sodium hydride (49 mg, 1.22 mmol, 60% in mineral oil). After 10 min of stirring, 2-[4-(2-trifluoromethylphenoxy)piperidin-1-yl]-7-chloro-5-methyl[1,3]thiazolo[4,5-d]pyrimidine (260 mg, 0.61 mmol) was added and the reaction heated to 120° C. for 16 h. The reaction mixture was quenched by dropwise addition of saturated aqueous NH₄Cl and concentrated. Purification by column chromatography through silica gel, eluting with 70% EtOAc in hexanes to 100% EtOAc to 10% EtOH in EtOAc as a gradient, afforded the title compound.

### Step 4: ({2-[4-(2-Trifluoromethylphenoxy)piperidin-1-yl]-5-methyl[1,3]thiazolo[4-5,d]pyrimidin-7-yl}oxy)acetic acid

Into a 10 mL round-bottom flask equipped with a magnetic stirbar was added ethyl ({2-[4-(2-trifluoromethylphenoxy)piperidin-1-yl]-5-methyl[1,3]thiazolo[4-5,d]pyrimidin-7-yl}oxy)acetate (88 mg, 0.18 mmol), methanol (1.0 mL) and 1M aqueous sodium hydroxide solution (350 uL, 0.35 mmol). The reaction mixture was stirred at 25° C. for 16 h. The cooled reaction mixture was concentrated and the residue poured into a 125 mL separatory funnel containing pH 5 buffer (KH₂PO₄, 75 mL) and the mixture was extracted with ethyl acetate (3×30 mL). The combined organic layers were washed with brine, dried over MgSO₄, filtered and the solvent was evaporated under reduced pressure. Purification by column chromatography through silica gel, eluting with 100% CH₂Cl₂ to 90:10 CH₂Cl₂:EtOH, gave the title compound.

¹H NMR (400 MHz, d₆-acetone): δ 7.68-7.61 (m, 2H), 7.40 (d, J=8.5 Hz, 1H), 7.11 (t, J=7.5 Hz, 1H), 4.99 (br s, 1H), 4.66 (br s, 2H), 3.76 (br s, 4H), 2.44 (s, 3H), 2.10-2.05 (m, 2H), 1.89-1.80 (m, 2H) ppm.

### Example of a Pharmaceutical Formulation

As a specific embodiment of an oral composition of a compound of the present invention, 50 mg of the compound of any of the Examples is formulated with sufficient finely divided lactose to provide a total amount of 580 to 590 mg to fill a size O hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 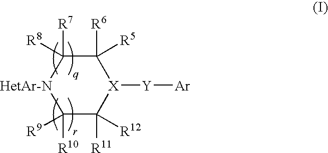
or a pharmaceutically acceptable salt thereof; wherein
HetAr is a fused heteroaromatic ring selected from the group consisting of: 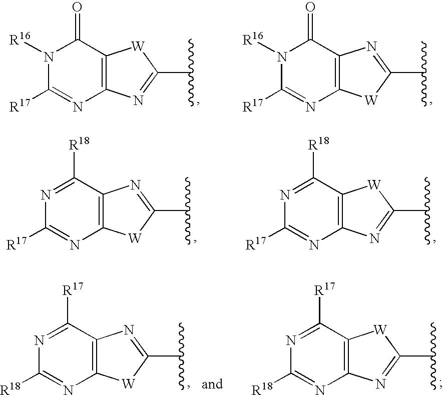
q is 0 or 1;
r is 0 or 1;
W is O, S, or NR¹⁵;
X—Y is N—C(O), CR¹⁴—O, CR¹⁴—S(O)₀₋₂, or CR¹³—CR¹R²;
Ar is phenyl, naphthyl, or heteroaryl optionally substituted with one to five R³ substituents;
R¹ and R² are each independently hydrogen or C₁₋₃ alkyl, wherein alkyl is optionally substituted with one to three substituents independently selected from fluorine and hydroxy;
each R³ is independently selected from the group consisting of:
C₁₋₆ alkyl,
C₂₋₆ alkenyl,
(CH₂)ₙ-phenyl,
(CH₂)ₙ-naphthyl,
(CH₂)ₙ-heteroaryl,
(CH₂)ₙ-heterocyclyl,
(CH₂)ₙC₃₋₇ cycloalkyl,
halogen,
nitro,
(CH₂)ₙOR⁴,
(CH₂)ₙN(R⁴)₂,
(CH₂)ₙC≡N,
(CH₂)ₙCO₂R⁴,
(CH₂)ₙNR⁴SO₂R⁴
(CH₂)ₙSO₂N(R⁴)₂,
(CH₂)ₙS(O)₀₋₂R⁴,
(CH₂)ₙNR⁴C(O)N(R⁴)₂,
(CH₂)ₙC(O)N(R⁴)₂,
(CH₂)ₙNR⁴C(O)R⁴,
(CH₂)ₙNR⁴CO₂R⁴,
(CH₂)ₙC(O)R⁴,
O(CH₂)ₙC(O)N(R⁴)₂,
(CH₂)ₛ-Z-(CH₂)ₜ-phenyl,
(CH₂)ₛ-Z-(CH₂)ₜ-naphthyl,
(CH₂)ₛ-Z-(CH₂)ₜ-heteroaryl,
(CH₂)ₛ-Z-(CH₂)ₜ-heterocyclyl,
(CH₂)ₛ-Z-(CH₂)ₜ—C₃₋₇ cycloalkyl,
(CH₂)ₛ-Z-(CH₂)ₜ—OR⁴,
(CH₂)ₛ-Z-(CH₂)ₜ—N(R⁴)₂,
(CH₂)ₛ-Z-(CH₂)ₜ—NR⁴SO₂R⁴,
(CH₂)ₛ-Z-(CH₂)ₜ—C≡N,
(CH₂)ₛ-Z-(CH₂)ₜ—CO₂R⁴,
(CH₂)ₛ-Z-(CH₂)ₜ—SO₂N(R⁴)₂,
(CH₂)ₛ-Z-(CH₂)ₜ—S(O)₀₋₂R⁴,
(CH₂)ₛ-Z-(CH₂)ₜ—NR⁴C(O)N(R⁴)₂,
(CH₂)ₛ-Z-(CH₂)ₜ—C(O)N(R⁴)₂,
(CH₂)ₛ-Z-(CH₂)ₜ—NR⁴C(O)R⁴,
(CH₂)ₛ-Z-(CH₂)ₜ—NR⁴CO₂R⁴,
(CH₂)ₛ-Z-(CH₂)ₜ—C(O)R⁴,
CF₃,
CH₂CF₃,
OCF₃, and
OCH₂CF₃;
in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;
Z is O, S, or NR⁴;
each R⁴ is independently selected from the group consisting of
hydrogen,
C₁₋₆ alkyl,
(CH₂)ₘ-phenyl,
(CH₂)ₘ-heteroaryl,
(CH₂)ₘ-naphthyl, and
(CH₂)ₘC₃₋₇ cycloalkyl;
wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁-—4 alkyl;
R⁵, R⁶, R⁷, R⁸, R⁹, R¹⁰, R¹¹, and R¹² are each independently hydrogen, fluorine, or C₁₋₃ alkyl, wherein alkyl is optionally substituted with one to three substituents independently selected from fluorine and hydroxy;
R¹³ is hydrogen, C₁₋₃ alkyl, fluorine, or hydroxy;
each R¹⁴ is independently hydrogen or C₁₋₃ alkyl;
R¹⁵ is selected from the group consisting of hydrogen, C₁₋₄ alkyl, C₁₋₄ alkylcarbonyl, aryl-C₁₋₂ alkylcarbonyl, arylcarbonyl, C₁₋₄ alkylaminocarbonyl, C₁₋₄ alkylsulfonyl, arylsulfonyl, aryl-C₁₋₂ alkylsulfonyl, C₁₋₄ alkyloxycarbonyl, aryloxycarbonyl, and aryl-C₁₋₂ alkyloxycarbonyl;
R¹⁶ is hydrogen or C₁₋₃ alkyl optionally substituted with one to five fluorines;
R¹⁷ is selected from the group consisting of:
—(CH₂)ᵥC(O)R^{a},
—(CH₂)_{y}-T-(CH₂)_{z}C(O)R^{a},
—(CH₂)_{y}-T-(CH₂)_{z}SO₃H,
—(CH₂)_{y}-T-(CH₂)_{w}-phenyl,
—(CH₂)_{y}-T-(CH₂)_{w}-heteroaryl, 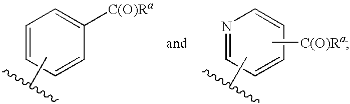
wherein phenyl and heteroaryl are optionally substituted with one to two substituents independently selected from halogen, C₁₋₄ alkyl, —(CH₂)_{X}C(O)R^{a}, and —CH═CHC(O)R^{a}; wherein any methylene (CH₂) carbon atom in R¹⁷ is optionally substituted with one to two groups independently selected from amino, carboxy, fluorine, hydroxy, and C₁₋₄ alkyl; or two substituents when on the same methylene (CH₂) group are taken together with the carbon atom to which they are attached to form a cyclopropyl group;
T is O, S, or NR¹⁴;
R^{a} is —OH, —OC₁₋₄ alkyl, —NH₂, —NHSO₂C₁₋₄ alkyl, —NHSO₂C₃₋₆ cycloalkyl, or —NHSO₂CH₂C₃₋₆ cycloalkyl;
R¹⁸ is selected from the group consisting of:
amino,
halogen,
C₁₋₄ alkoxy, optionally substituted with hydroxy or carboxy,
C₁₋₄ alkylthio, optionally substituted with hydroxy or carboxy,
C₁₋₄ alkylamino,
di-(C₁₋₄ alkyl)amino,
arylamino,
aryl-C₁₋₂ alkylamino,
C₁₋₄ alkylcarbonylamino,
aryl-C₁₋₂ alkylcarbonylamino,
arylcarbonylamino,
C₁₋₄ alkylaminocarbonylamino,
C₁₋₄ alkylsulfonylamino,
arylsulfonylamino,
aryl-C₁₋₂ alkylsulfonylamino,
C₁₋₄ alkyloxycarbonylamino,
aryloxycarbonylamino, and
aryl-C₁₋₂ alkyloxycarbonylamino;
each m is independently an integer from 0 to 2;
each n is independently an integer from 0 to 2;
each s is independently an integer from 1 to 3;
each t is independently an integer from 1 to 3;
v is an integer from 0 to 4;
w is an integer from 0 to 2;
z is 1 or 2;
each x is an integer from 0 to 2; and
each y is 0 or 1.

2. The compound of claim 1 wherein q and r are both 1.

3. The compound of claim 1 wherein X—Y is CR¹⁴—O.

4. The compound of claim 3 wherein R¹⁴ is hydrogen and Ar is phenyl substituted with one to three R³ substituents.

5. The compound of claim 1 wherein R⁵, R⁶, R⁷, R⁸, R⁹, R¹⁰, R¹¹, and R¹² are each hydrogen.

6. The compound of claim 1 wherein HetAr is

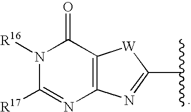

7. The compound of claim 6 wherein W is S and R¹⁶ is hydrogen.

8. The compound of claim 6 wherein R¹⁷ is —(CH₂)ᵥC(O)R^{a} wherein R^{a} is —OH or —OC₁₋₄ alkyl and v is an integer from 1 to 3.

9. The compound of claim 8 wherein v is 2.

10. The compound of claim 6 wherein R¹⁷ is —(CH₂)_{y}—S—(CH₂)C(O)R^{a} wherein R^{a} is —OH or —OC₁₋₄ alkyl and y is 0 or 1.

11. The compound of claim 6 wherein R¹⁷ is —(CH₂)_{y}-T-(CH₂)_{w}-pyridyl or —(CH₂)_{y}-T-(CH₂)_{w}-phenyl wherein
y is 0 or 1;
w is 0 or 1;
T is O or S; and
phenyl and pyridyl are substituted with one substituent selected from —(CH₂)ₓC(O)R^{a} and —CH═CHC(O)R^{a} wherein R^{a} is —OH or —OC₁₋₄ alkyl and x is 0 or 1.

12. The compound of claim 1 wherein Ar is phenyl substituted with one to two substituents independently selected from the group consisting from C₁₋₄ alkyl, halogen, CF₃, and phenyl optionally substituted with one to two substituents independently selected from the group consisting of halogen, hydroxy, C₁₋₄ alkyl, trifluoromethyl, and C₁₋₄ alkoxy.

13. The compound of claim 1 of the structural formula (II): 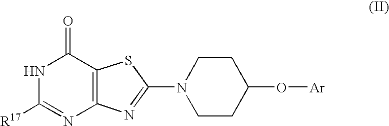
wherein Ar is phenyl substituted with one to two substituents independently selected from the group consisting from C₁₋₄ alkyl, halogen, CF₃, and phenyl optionally substituted with one to two substituents independently selected from the group consisting of halogen, hydroxy, C₁₋₄ alkyl, trifluoromethyl, and C₁₋₄ alkoxy;
R¹⁷ is selected from the group consisting of:
—(CH₂)ᵥC(O)R^{a},
—(CH₂)_{y}—S—CH₂C(O)R^{a},
—(CH₂)_{y}-T-(CH₂)_{w}-pyridyl, and
—(CH₂)_{y}-T-(CH₂)_{w}-phenyl;
T is O or S; and phenyl and pyridyl are substituted with one substituent selected from —(CH₂)ₓC(O)R^{a} and —CH═CHC(O)R^{a}; and wherein R^{a} is —OH or —OC₁₋₄ alkyl; v is an integer from 1 to 3; y is 0 or 1; w is 0 or 1; and x is an integer from 0 to 2.

14. The compound of claim 1 of structural formula (III): 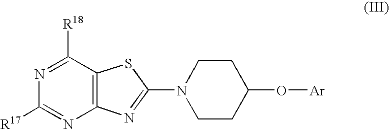
wherein Ar is phenyl substituted with one to two substituents independently selected from the group consisting from C₁₋₄ alkyl, halogen, CF₃, and phenyl optionally substituted with one to two substituents independently selected from the group consisting of halogen, hydroxy, C₁₋₄ alkyl, trifluoromethyl, and C₁₋₄ alkoxy;
R¹⁸ is selected from the group consisting of
amino,
halogen,
C₁₋₄ alkoxy, optionally substituted with hydroxy or carboxy,
C₁₋₄ alkylthio, optionally substituted with hydroxy or carboxy,
C₁₋₄ alkylamino, and
di-(C₁₋₄ alkyl)amino;
R¹⁷ is selected from the group consisting of
—(CH₂)ᵥC(O)R^{a},
—(CH₂)_{y}—S—CH₂C(O)R^{a},
—(CH₂)_{y}-T-(CH₂)_{w}-pyridyl, and
—(CH₂)_{y}-T-(CH₂)_{w}-phenyl;
T is O or S; and phenyl and pyridyl are substituted with one substituent selected from —(CH₂)ₓC(O)R^{a} and —CH═CHC(O)R^{a}; and wherein R^{a} is —OH or —OC₁₋₄ alkyl; v is an integer from 1 to 3; y is 0 or 1; w is 0 or 1; and x is an integer from 0 to 2.

15. The compound of claim 5 which is selected from the group consisting of: 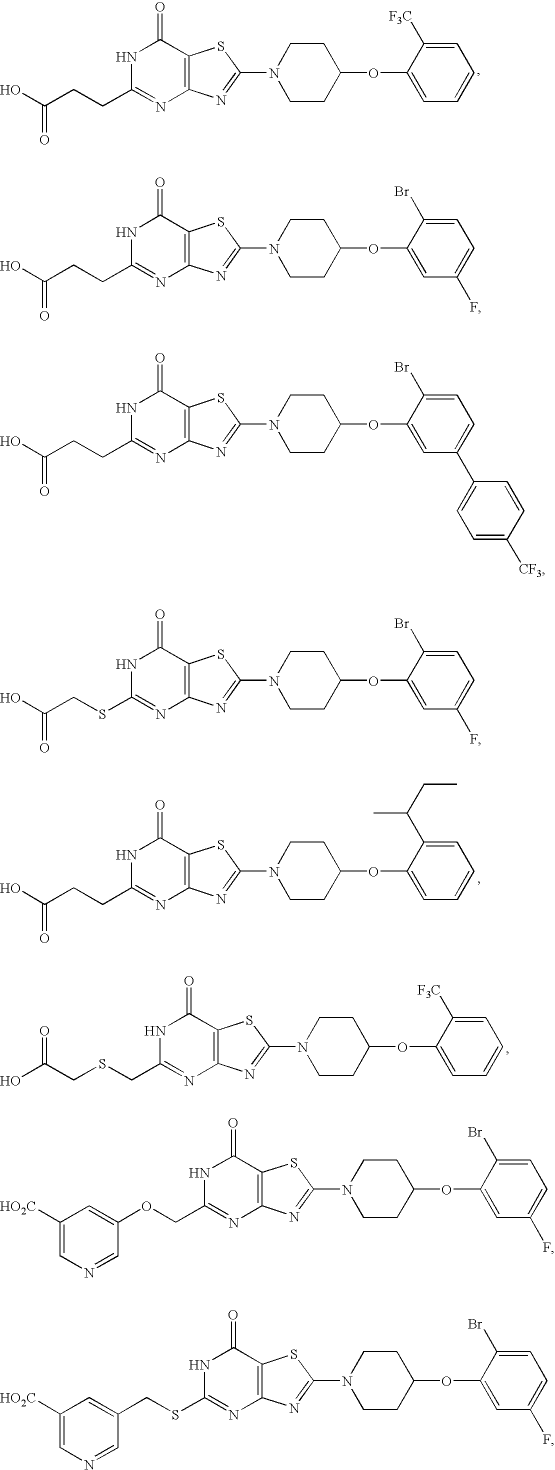 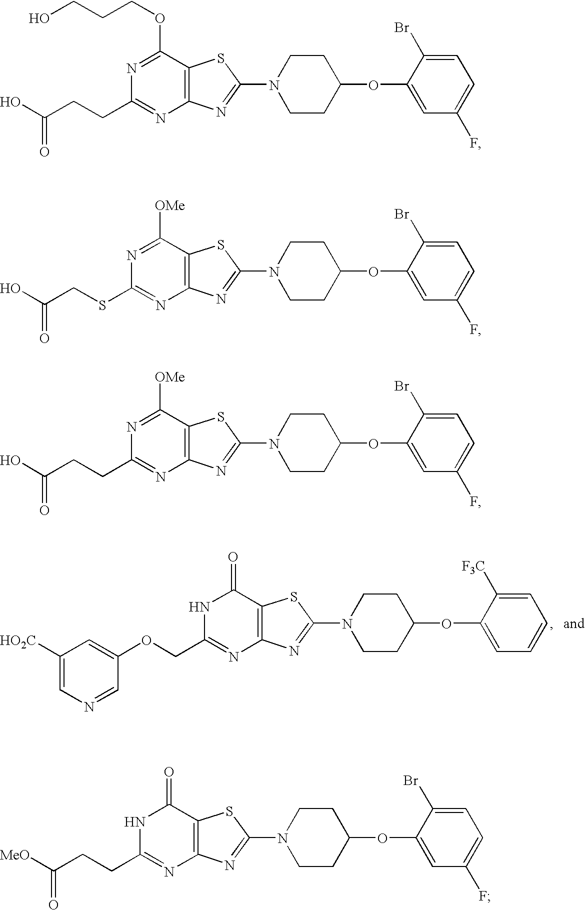
or a pharmaceutically acceptable salt thereof.

16. A pharmaceutical composition comprising a compound in accordance with claim 1 in combination with a pharmaceutically acceptable carrier.

17-21. (canceled)

22. A method for treating non-insulin dependent (Type 2) diabetes, insulin resistance, hyperglycemia, a lipid disorder, obesity, and fatty liver disease in a mammal in need thereof which comprises the administration to the mammal of a therapeutically effective amount of a compound of claim 1.

23. The method of claim 22 wherein said lipid disorder is selected from the group consisting of dyslipidemia, hyperlipidemia, hypertriglyceridemia, atherosclerosis, hypercholesterolemia, low HDL, and high LDL.
